(19) United States
(12) Patent Application Publication
(10) Pub. No.: US 2004/0162333 A1
(43) Pub. Date: Aug. 19, 2004

(54) Rapid absorption selective 5-HT agonist formulations

(76) Inventors: Naima Mezaache, McLean, VA (US); Djelila Mezaache, Laurel, MD (US); Steve Frisbee, Reston, VA (US); Paul Maes, Toronto (CA)

Correspondence Address: CROWELL & MORING LLP, INTELLECTUAL PROPERTY GROUP, P.O. BOX 14300, WASHINGTON, DC, 20044-4300, US

(21) Appl. No.: 10/779,784

(22) Filed: Feb. 18, 2004

Related U.S. Application Data
(60) Provisional application No. 60/447,741, filed on Feb. 19, 2003.

(51) Int. Cl. A61K 31/405

(52) U.S. Cl. 514/419

(57) ABSTRACT

The invention provides for a rapid absorption pharmaceutical composition comprising an effective amount of at least one selective 5-HT agonist, at least one spheronization aid and at least one solubility enhancer. The composition of the invention is incorporated into microparticles, which may be subsequently taste-masked and incorporated into a variety of dosage forms for administration to a patient suffering from migraine

### RELATED APPLICATIONS

This application claims the benefit of priority of U.S. Provisional Application No. 60/447,741 filed Feb. 19, 2003 the entire disclosure of which is incorporated herein by reference.

### FIELD OF THE INVENTION

The present invention relates to rapid absorption oral dosage pharmaceutical preparations comprising an effective amount of at least one selective 5-HT agonist for the treatment of migraine.

### BACKGROUND

Migraine is a common condition, affecting 15% to 20% of women and about half as many men in any given year. Prevalence is highest in the 25- to 44-year age group, when most individuals are employed. The US National Headache Foundation estimates that US businesses lose approximately $50 billion each year because of headache-related absenteeism, reduced employee productivity, and medical expenses. Migraine is the most common cause of severe recurring headache and accounts for the bulk of this financial loss. A recent estimate of the burden of migraine in the US showed that employers lose about $13 billion annually because of missed workdays and impaired work function. As is the case with many medical and non-medical situations, most headaches and lost workdays are borne by a minority of the individual migraine sufferers.

While migraine headache is a chronic condition with potentially debilitating effects, prophylactic and symptomatic treatments are available. In particular, the development of selective serotonin agonists has been a tremendous breakthrough in the treatment of migraine headaches. The so called triptans are serotonin (5-hydroxytryptamine [5-HT])_{1B/1D} receptor-specific agonists that specifically abort migraine. Sumatriptan (Imitrex®, GlaxoSmithKline), the first triptan to be introduced, was synthesized in the 1980s and has been in clinical use for more than a decade. Other triptans now available include zolmitriptan (Zomig®, Astra Zeneca), naratriptan (Amerge®, GlaxoSmithKline), rizatriptan (Maxalt®, Merck), almotriptan (Axert®, Pharmacia), and frovatriptan (Frovelan®, Elan). Eletriptan® (Relpax, Pfizer) is currently before the US Food and Drug Administration. Other treatments are available for migraine but often have accompanying adverse effects that prevent individuals from returning to their normal activities.

The time to peak effect for the various commercially available oral triptans has been reported to be as follows (Johnson, K. Migraine Therapy: balancing efficacy and safety with quality of life and cost, Formulary; 2002:37, pp. 634-644):

| | Time to Peak Effect |
|---|---|
| | (Tₘₐₓ)(hours) |
| Sumatriptan | 2.5 |
| Zolmitriptan (Zomig) | 2 |
| Naratriptan (Amerge) | 2-3 |
| Rizatriptan (Maxalt) | 1-1.5 |
| Almotriptan (Axert) | 1-3 |
| Frovatriptan (Frova) | 2-4 |

As mentioned above, migraine headache is a chronic condition with potentially debilitating effects. Accordingly, it would be advantageous to develop a triptan dosage form that could further increase the absorption rate of the triptan into the blood stream of a migraine patient thereby increasing the possibility of more rapid onset of action of the drug.

Oral administration of drugs, including the triptans, is currently the most popular route of administration of drugs. Also, solid oral delivery systems do not require sterile conditions and are, therefore, less expensive to manufacture. A constant problem, however, in orally medicating patients is their frequent inability or unwillingness to swallow a solid dosage form. In addition there is often a lack of acceptance of orally disintegrating tablets or chewable tablets that have a pronounced bitterness.

Oral fast-dispersing dosage forms, also known as fast dissolve, rapid dissolve, rapid melt and quick disintegrating tablets, are gaining popularity as the oral dosage form of choice. This is particularly true for pediatric and geriatric patients who frequently have difficulty swallowing conventional solid-dosage forms. In addition, for many medicaments, the act of swallowing the medicament often requires fluids that increase gastric volume and the likelihood of nausea and vomiting. This occurs more often in migraine patients. Perhaps the biggest advantage of oral fast-dispersing dosage forms is that the solid dosage form dissolves or disintegrates quickly in the oral cavity, resulting in a solution or suspension without the need for the administration of fluid. Accordingly, the patient can administer the dosage form as soon as symptoms are felt. Oral fast-dispersing dosage forms and processes for making same are well known in the art and are described for example in U.S. Pat. Nos. 4,616,047, 4,642,903, 5,073,374, 5,112,616, 5,178,878, 5,188,825, 5,219,574, 5,223,264, 5,401,513, 5,446,464, 5,464,632, 5,503,846, 5,567,439, 5,576,014, 5,587,172, 5,587,180, 5,595,761, 5,607,697, 5,613,023, 5,622,719, 5,635,210, 5,776,491, 5,807,576, 5,807,577, 5,807,578, 5,827,541, 5,851,553, 5,866,163, 5,869,098, and 5,871,781.

Given that the triptans have been clinically proven to be efficacious for the treatment of migraine, it would be advantageous to develop a formulation from which the triptan will be absorbed at a significantly faster rate and possibly achieve a much more rapid onset of action for a given dose than that currently available with the commercially available triptan products. One way of achieving this would be to develop a rapid absorption oral fast-dispersing dosage form of the triptans. Further, it would be preferable if the dosage form comprising the rapid absorption composition were ingestible without water. This is particularly important because migraine sufferers must dose themselves as soon as possible once an aura or migraine occurs.

Conventional and oral fast-dispersing dosage forms comprising sumatriptan have been described in various patents and published patent applications. For example, International Patent Publication No. WO 01/39836 describes a novel freeze-dried pharmaceutical composition useful for the treatment of migraine and associated symptoms at a reduced total dose of active substance than required for oral administration in the form of a tablet containing a porous matrix net work of a water soluble or water dispersible carrier material together with the pharmaceutically active substance and other excipients.

U.S. Pat. No. 6,383,471 describes pharmaceutical compositions capable of solubilizing therapeutically effective amounts of ionizable hydrophobic therapeutic agents with the aim of maintaining the solubilized ionizable hydrophobic therapeutic agent in solubilized form upon administration to a patient and/or improving the delivery of the therapeutic agent to the absorption site. Among the list of therapeutic agents contemplated is sumatriptan. Similarly, International Patent Publication No. WO 01/37808 is directed to solid pharmaceutical compositions for improving delivery of a wide variety of pharmaceutical active ingredients contained therein. Sumatriptan is one of the pharmaceutically active ingredients contemplated.

International Patent Publication No. WO 92/15295 describes a pharmaceutical composition for oral administration comprising a film-coated dosage form including sumatriptan or a pharmaceutically acceptable salt or solvate thereof as active ingredient.

International Patent Publication No. WO 98/42344 describes a pharmaceutical composition for oral administration comprising a carrier and, as an active ingredient, a 5-HT₁agonist, characterized in that the composition is formulated to reduce pre-systemic metabolism of the 5-HT₁ agonist. In other words, the composition is formulated to promote pre-gastric absorption of the 5-HT₁agonist and hence increase the bioavailability of the drug.

International Patent Publication No. WO98/02187 describes a formulation for enhancing the penetration of a drug, including sumatriptan, thereby increasing the bioavailability of the drug.

Oral fast-dispersing dosage forms are currently available for rizatriptan (Maxalt-MLT) and zolmitriptan (Zomig-ZMT). Unfortunately, however, the Tₘₐₓ (time to maximum plasma concentration) for both these products is slower than their respective conventional oral dosage forms. For example, the Tₘₐₓ for the conventional rizatriptan oral dosage form, Maxalt, is approximately 1-1.5 hours, whereas the Tₘₐₓ for the oral fast-dispersing dosage form, Maxalt-MLT, averages 1.6 to 2.5 hours. Similarly, the Tₘₐₓ for the conventional zolmitriptan oral dosage form, Zomig is about 1.5 hours, whereas, the Tₘₐₓ for the oral fast-dispersing dosage form, Zomig-ZMT, is about 3 hours.

The above-described formulations appear to be directed to either improving delivery of a triptan to the site of absorption or enhancing penetration of the drug, thereby increasing its bioavailability. None of the above formulations are directed to increasing the rate of absorption of the drug, which would potentially bring about a faster onset of action. Thus, there still exists a need for a rapid absorption composition comprising at least one selective 5-HT agonist for the treatment of migraine in the form of an oral fast-dispersing dosage form.

Definitions

The phrase “oral fast-dispersing dosage form” as used herein is interchangeable with fast-dissolve, rapid dissolve, rapid melt, quick disintegrating, orally dispersible, fast disperse orally disintegrating tablets, and the like. All such dosage forms are typically in the form of tablets and are adapted to dissolve, disperse or disintegrate rapidly in the oral cavity, resulting in a solution or suspension without the need for the administration of a fluid. Any such dosage form is consistent with the objects of the invention. It is preferred that the dosage form of the invention dissolve, disintegrate or disperse in 50 seconds or less, preferably in 30 seconds or less and most preferably in 20 seconds or less.

As used herein, “rapid absorption” means a lower T₅₀ with an equal or higher Cₘₐₓ, of an oral dosage form of the invention when compared to a currently marketed oral triptan product, but having an area under the plasma-concentration time curve (AUC) that is equivalent to the currently marketed oral triptan product. Cₘₐₓ is the observed maximum plasma concentration and can be measured after a single-dose or steady state of the triptan for every dose given. Wagner-Nelson deconvolution defines T₅₀ as the time taken for 50% of the drug to be absorbed into the system. The reader is referred to Gibaldi M. and Perrier D. Pharmacokinetics. New York: Marcel Dekker, Inc. 1982 for a detailed discussion of Wagner-Nelson deconvolution analysis. The AUC, or the Area Under the Curve, of the pharmacokinetic profile, signifies the extent of absorption of the drug.

The selective 5-HT agonist as used herein is the pharmaceutically acceptable salt of the triptan. As used herein the term “pharmaceutically acceptable salt” includes salts that are physiologically tolerated by a patient. Such salts are typically prepared from inorganic acids or bases and/or organic acids or bases. Examples of such acids and bases are well known to those of ordinary skill in the art. The invention in particular contemplates the use of the selective 5-HT agonist sumatriptan succinate, although as mentioned above the use of the sumatriptan base, without an associated salt is within the scope of the invention.

An effective amount of a selective 5-HT agonist is specifically contemplated. By the term “effective amount,” it is understood that “a pharmaceutically effective amount” is contemplated. A “pharmaceutically effective amount” is the amount or quantity of the selective 5-HT agonist, which is sufficient to elicit an appreciable biological response when administered to a patient. It will be appreciated that the amount of the selective 5-HT agonist employed in the composition of the invention will depend on the particular triptan used. Furthermore, the precise therapeutic dose of the active ingredient will depend on the age and condition of the patient and the nature of the condition to be treated and will be at the ultimate discretion of the attendant physician.

### SUMMARY OF THE INVENTION

The first aspect of the invention is a rapid absorption composition comprising at least one selective 5-HT agonist, at least one spheronization aid and at least one solubility enhancer.

In one embodiment the composition of the invention is incorporated into microparticles. The microparticles can be further incorporated into any dosage form in which microparticles comprising the composition of the invention can be incorporated into. The dosage form preferably takes the form of a fast-dispersing direct compression non-cushioning matrix tablet.

The selective 5-HT agonist used herein is preferably sumatriptan and ranges from about 1% to about 60%, preferably from about 20% to about 50% and most preferably about 30% to about 40% by weight of the microparticle.

The preferred spheronization aid is glyceryl palmitostearate. However, other spheronization aids known in the art are operable. The amount of spheronization aid comprising the microparticle is in the range from about 5% to about 90%, preferably from about 15% to about 75%, and most preferably from about 25% to about 45% by weight of a microparticle.

The preferred solubility enhancers are macrogol fatty acid esters selected from those containing from about 30 to about 35 oxyethylene groups. The most preferred macrogol fatty acid esters are sold under the trade name Gelucire® 50/13 or Gelucire® 44/14. The solubility enhancer(s) comprising the microparticles are in the range of from greater than about 0% to about 95%, preferably from about 1% to about 50% and most preferably from about 5% to about 35% by weight of the microparticle.

It is preferred that the microparticles contain only the selective 5-HT agonist(s), spheronization aid(s) and solubility enhancer(s). However, other excipients consistent with the objects of the invention are not precluded from use. Such excipients can include diluents (or fillers), disintegrants, binders, glidants, lubricants, antiadherents, sorbents, flavourants, colourants, etc.

It is preferred that the microparticles comprising the rapid absorption composition are manufactured using the assignee's proprietary CEFORM™ technology under liquiflash conditions, however other methods of making the microparticles are not precluded.

It is preferred that the microparticles are coated with at least one taste-masking coating. Useful taste-masking coatings include a combination of hydrophobic and hydrophilic polymers. The preferred hydrophobic polymer is Ethylcellulose E45 and the preferred hydrophilic polymer is Povidone K30 in a ratio of 7:3 respectively.

The microparticles comprising the composition of the invention are intended to be used in the manufacture of medicaments for the treatment of migraine.

In another aspect of the invention the microparticles comprising the composition of the invention are incorporated into a fast-dispersing direct compression non-cushioning matrix dosage form.

The non-cushioning matrix is comprised of a linear polyol and/or lactose or maltose sugars, and optionally an inorganic salt, a cellulose or cellulose derivative, or a mixture thereof.

It is preferred that the linear polyol is a directly compressible form of mannitol. The linear polyol(s) is present in an amount from about greater than 0% to about 85%, preferably from about 20% to about 60% and most preferably from about 40% to about 50% by weight of the dosage form.

The preferred optional inorganic salt is a directly compressible form of dibasic anhydrous calcium phosphate. The directly compressible inorganic salt comprising the non-cushioning matrix may be present in the range from about 0% to about 50%, preferably from about 5% to about 30% and most preferably from about 7% to about 15% by weight of the dosage form.

The preferred optional cellulose is directly compressible microcrystalline cellulose. However, other powdered or directly compressible forms of cellulose or cellulose derivatives are not precluded. The directly compressible celluloses may be present in the non-cushioning matrix excipient mass in the range from about 0% to about 40%, preferably from about 5% to about 30% and most preferably from about 10% to about 20% by weight of the fast-dispersing direct compression non-cushioning matrix dosage form.

It is also preferred that the dosage form comprise a superdisintegrating agent. Preferably, this agent is crospovidone, but does not preclude other superdisintegrating agents or agents which assist in the fast dispersal of the dosage form.

In one aspect of the invention the dosage form comprising the microparticles comprises a composition with a low macrogol fatty acid ester content. This composition, when administered to a patient in need of such administration exhibits a blood absorption profile such that after about 0.5 hours at least about 15% of the sumatriptan is absorbed, after about 0.75 hours at least about 35% of the sumatriptan is absorbed, after about 1 hour at least about 50% of the sumatriptan is absorbed, after about 1.5 hours at least about 70% of the sumatriptan is absorbed, after about 2 hours at least about 80% of the sumatriptan is absorbed, after about 4 hours at least about 90% of the sumatriptan is absorbed, and after about 6 hours at least about 95% of the sumatriptan is absorbed into the blood stream of the patient.

In another aspect of the invention the dosage form comprising the microparticles with the low macrogol fatty acid ester content provides a Tₘₐₓ from about 1 hour to about 3 hours and a Cₘₐₓ of about 15 ng/ml to about 46 ng/ml sumatriptan with a mean Tₘₐₓ of about 1.7 hours and a mean Cₘₐₓ of about 28 ng/ml sumatriptan in the blood after administration of a 50 mg sumatriptan dosage form to a patient in need of such administration. This dosage form exhibits an AUC₍₀₋ₜ₎ from about 69 ng.hr/ml to about 163 ng.hr/ml with a mean AUC₍₀₋ₜ₎ of about 109 ng.hr/ml.

In one aspect of the invention, the dosage form comprising the microparticles comprises a composition with a high macrogol fatty acid ester content when administered to a patient in need of such administration and exhibits a blood absorption profile such that after about 0.5 hours at least about 20% of the sumatriptan is absorbed, after about 0.75 hours at least about 40% of the sumatriptan is absorbed, after about 1 hour at least about 55% of the sumatriptan is absorbed, after about 1.5 hours at least about 76% of the sumatriptan is absorbed, after about 2 hours at least about 80% of the sumatriptan is absorbed, after about 4 hours at least about 90% of the sumatriptan is absorbed, and after about 6 hours at least about 95% of the sumatriptan is absorbed into the blood stream of the patient.

In another aspect of the invention the dosage form comprising the microparticles with the high macrogol fatty acid ester content provides a Tₘₐₓ from about 0.75 hours to about 2 hours and a Cₘₐₓ of about 14 ng/ml to about 46 ng/ml sumatriptan with a mean Tₘₐₓ of about 1.6 hours and a mean Cₘₐₓ of about 27 ng/ml sumatriptan in the blood after administration of a 50 mg sumatriptan dosage form to a patient in need of such administration. This dosage form exhibits an AUC₍₀₋ₜ₎ from about 60 ng.hr/ml to about 165 ng.hr/ml with a mean AUC₍₀₋ₜ₎ of about 110 ng.hr/ml.

In this invention, it has been found that the CEFORM™ technology for manufacturing the microparticles comprising the composition of the invention combined with a specifically formulated barrier layer successfully masked the bitter taste of the selective 5-HT agonist sumatriptan. Considering the small size and bitter taste of the microparticles, this surprisingly, occurred at low coating levels of about 20% by weight of each microparticle.

Bioavailability studies confirmed that the formulations of the invention were bioequivalent to the prior art product Imitrex®. However, surprisingly, both formulations with low and high macrogol fatty acid ester content exhibited a significantly faster absorption rate than the reference Imitrex® product. This was an unexpected result.

### BRIEF DESCRIPTION OF THE FIGURES

The present invention will be further understood from the following detailed description with references to the following drawings.

FIG. 1 is a graph illustrating the dissolution profile of coated and uncoated low macrogol fatty acid ester content microparticles according to an embodiment of the invention.

FIG. 2 is a graph illustrating the dissolution profile of coated and uncoated high macrogol fatty acid ester content microparticles according to an embodiment of the invention.

FIG. 3 is a graph illustrating the comparison of dissolution profiles of direct compression non-cushioning matrix tablets comprising microparticles having the 5-HT agonist sumatriptan, at least one spheronization aid and a high or low macrogol fatty acid ester content made according to an embodiment of the invention and the dissolution profile of the prior art Imitrex® tablet.

FIG. 4 is a graph illustrating the dissolution profile of a direct compression non-cushioning matrix tablet comprising microparticles having the 5-HT agonist sumatriptan, at least one spheronization aid and a high macrogol fatty acid ester content made according to an embodiment of the invention.

FIG. 5A is a graph illustrating the mean in vivo sumatriptan plasma concentrations following administration of a single-dose sumatriptan 50 mg direct compression non-cushioning matrix tablet made according to an embodiment of the invention over a period of 12 hours.

FIG. 5B is a graph illustrating the differences between the graph in FIG. 5A to the prior art Imitrex® tablet.

FIG. 5C is a graph further illustrating the differences in the mean in vivo succinate plasma concentrations of FIG. 5B over the first 2 hours after administration.

FIG. 6A is a graph illustrating the mean in vivo absorption profile of sumatriptan following administration of a single-dose sumatriptan 50 mg direct compression non-cushioning matrix tablet made according to an embodiment of the invention over a period of 12 hours.

FIG. 6B is a graph illustrating the differences between the graph in FIG. 6A to the absorption profile of the prior art Imitrex® tablet.

FIG. 6C is a graph further illustrating the differences between the absorption profiles of FIG. 6B over the first 4 hours after administration.

FIG. 7A is a graph illustrating the mean in vivo sumatriptan plasma concentrations following administration of a single-dose sumatriptan 50 mg direct compression non-cushioning matrix tablet made according to an embodiment of the invention over a period of 12 hours.

FIG. 7B is a graph illustrating the differences between the graph in FIG. 7A to the prior art Imitrex® tablet.

FIG. 7C is a graph further illustrating the differences in the mean in vivo sumatriptan plasma concentrations of FIG. 7B over the first 2 hours after administration.

FIG. 8A is a graph illustrating the mean in vivo absorption profile of sumatriptan following administration of a single-dose sumatriptan 50 mg direct compression non-cushioning matrix tablet made according to an embodiment of the invention over a period of 12 hours.

FIG. 8B is a graph illustrating the differences between the graph in FIG. 8A to the absorption profile of the prior art Imitrex® tablet.

FIG. 8C is a graph further illustrating the differences between the absorption profiles of FIG. 8B over the first 4 hours after administration.

FIG. 9 is a graph illustrating the dissolution profile of a conventional non-cushioning matrix tablet comprising microparticles having the 5-HT agonist sumatriptan, at least one spheronization aid and at least one solubility enhancer.

### DETAILED DESCRIPTION OF THE INVENTION

This invention relates to rapid absorption compositions comprising an effective amount of at least one selective 5-HT agonist for the treatment of migraine, at least one solubility enhancer, and at least one spheronization aid. The rapid absorption composition of the invention is incorporated into microparticles, which due to their spherical nature and robustness, can be used in a number of different delivery systems including but not limited to fast-dispersing direct compression non-cushioning matrix tablets, a fast-dispersing direct compression cushioning matrix tablets, direct compression non-cushioning matrix tablets, direct compression cushioning matrix tablets, capsules, buccal tablet, sachets and the like.

I. Microparticles

The rapid absorption composition of the invention takes the form of microparticles. The microparticles of the invention comprise an effective amount of at least one selective 5-HT agonist, at least one spheronization aid and at least one solubility enhancer. The term “microparticles” as used herein is interchangeable with the terms “microspheres”, “spherical particles” and “microcapsules”.

The selective 5-HT agonist used herein can be selected from the group of selective 5-HT agonists, which include but are not limited to sumatriptan, zolmitriptan, rizatriptan, naratriptan, frovatriptan, eletriptan, and almotriptan. Combinations of selective 5-HT agonists may be used providing the combinations have been shown not to have a synergistic effect and thereby cause a serious vasospastic adverse event.

The preferred selective 5-HT agonist is sumatriptan. The amount of selective 5-HT agonist comprising the microparticles is in the range of from about 1% to about 60%, preferably from about 20% to about 50% and most preferably about 30% to about 40% by weight of a microparticle.

Spheronization aid(s) used herein are materials, which help the drug-containing mix to form robust durable microparticles. Some examples of materials useful as spheronization aids include, but are not limited to distilled monoglycerides, glyceryl behenate, glyceryl palmitostearate, hydrogenated vegetable oils, sodium lauryl sulfate, polyoxyethylene ethers, cetostearyl alcohol, waxes and wax-like materials. Certain thermo-plastic or thermo-softening polymers may also function as spheronization aids. Non-limiting examples of such thermo-plastic or thermo-softening polymers include povidone, cellulose ethers, polymethacrylates and polyvinylalcohols. Mixtures of spheronization aids can also be used. The preferred spheronization aid is glyceryl palmitostearate and is sold under the trade name Precirol® ato 5. Precirol® ato 5 is synthesized by esterification of glycerol by palmitostearic acid (C16-C18 fatty acid). The raw materials used are of strictly vegetable origin and the reaction process involves no catalyst. The product is then atomized by spray cooling. Precirol® ato 5 is composed of mono-, di and triglycerides of palmitostearic acid, the diester fraction being predominant. The spheronization aid(s) is present in an amount ranging from about 5% to about 90%, preferably from about 15% to about 75% and most preferably from about 25% to about 45% by weight of a microparticle.

Solubility enhancers are surfactants and other materials included in the microparticles to assist in the dissolution of a drug. The ability of a surfactant to reduce the solid/liquid interfacial tension will permit fluids to wet the solid more effectively and thus aid the penetration of fluids into the drug-excipient mass to increase the dissolution rate and absorption rate of the drug. Some examples of the preferred materials useful as solubility enhancers include polyethylene glycol glyceryl esters (macrogol fatty acid esters), polyethylene glycol, polyethylene glycol derivatives of lipophilic molecules such as polyethylene glycol fatty acid esters, polyethylene glycol fatty alcohol ethers, polymeric surfactant materials containing one or more polyoxyalkylene blocks, such as poloxamers, and other polyoxyethylene/polyoxypropylene copolymers as well as sucrose ethers and esters. Combinations of solubility enhancers can be used. The macrogol fatty acid esters useful herein are selected from those containing from about 30 to about 35 oxyethylene groups. The preferred macrogol fatty acid esters are sold under the trade name Gelucire®, and includes but is not limited to Gelucire 50/13® or Gelucire 44/14®, with Gelucire 50/13® being the most preferred. The solubility enhancer(s) is present in an amount ranging from greater than about 0% to about 95%, preferably from about 1% to about 50% and most preferably from about 5% to about 35% by weight of a microparticle.

It is preferred that the microparticles contain only the selective 5-HT agonist(s), solubilizer(s) and spheronization aid(s). However, if necessary, additional excipients consistent with the objects of the invention may also be used. The additional excipients may be added to facilitate the preparation, patient acceptability and functioning of the dosage form as a drug delivery system. The other excipients can include, but are not limited to, diluents (or fillers), disintegrants, binders, glidants, lubricants, antiadherents, sorbents, flavourants, colourants, etc.

It is preferred that microparticles comprising the rapid absorption composition of the invention are manufactured using the applicant's proprietary CEFORM™ (Centrifugally Extruded & Formed Microspheres) technology, which is the simultaneous use of flash heat and centrifugal force, using proprietary designed equipment, to convert dry powder systems into microparticles of uniform size and shape. The microparticles of the invention are prepared by hot-melt encapsulation described in detail in U.S. Pat. Nos. 5,587,172, 5,616,344, and 5,622,719, which contents are wholly incorporated herein by reference. The process for manufacturing the microparticles of the invention are not limited to the CEFORM™ technology, and any other technology resulting in the formation of microparticles consistent with the objects of the invention may also be used.

Two fundamental processes are used to produce microparticles comprising the rapid absorption composition of the invention: (1) the encapsulation process and (2) the co-melt process. In the encapsulation approach, the process is conducted below the melting point of the drug. Therefore, the excipients are designed to melt and entrain the drug particles on passing through the apertures to form microparticles. The resulting microparticles contain the drug, in its native state, essentially enveloped by or as an intimate matrix with the resolidified excipients. In the co-melt approach, the process is conducted above the melting point of the drug. In this case, the drug and the excipients melt or become fluid simultaneously upon exposure to the heat. The molten mixture exits the head and forms microparticles, which cool as they fall to the bottom of the collection bin where they are collected.

It is preferred that the microparticles of the invention comprising the selective 5-HT agonist(s) are manufactured using the encapsulation approach, with at least one spheronizing agent, which also acts as a drug carrier, and at least one solubility enhancer. The encapsulation approach is favored because it is believed that the hydrophilic solubilizer(s) encapsulates the hydrophobic selective 5-HT agonist, thus aiding the solubility of the selective 5-HT agonist. In the encapsulation technique the excipient(s) which are chosen must a have a lower melting point than the drug with which they will be combined (158.4-159 reference: Merck Index, 12^{th} edition). Therefore, the spheronizing process can be performed at lower temperatures, than the melting point of the drug. This eliminates the risk of polymeric interconversion, which can occur when using processing temperatures close to the melting point.

The processing of the microparticles comprising the rapid absorption composition of the invention is carried out in a continuous fashion under” liquiflash conditions”. Liquiflash conditions are generally those under which the material, called a feedstock (a pre-blend of drug (selective 5-HT agonist) and excipients (solubilizing agent(s) and spheronization aid(s)) is fed into a spinning head. The spinning head is a multi-aperture production unit, which spins on its axis and is heated by electrical power. One particular head highly useful in making the microparticles comprising the rapid absorption composition of the invention is described in U.S. Pat. No. 5,458,823. The '823 patent describes a spinning head including a base and a cover. A plurality of closely spaced heating elements are positioned between the base and the cover, forming a barrier through which the material to be processed passes. In use, the head rotates and the heating elements are heated to temperatures that bring about liquiflash conditions in the feedstock being processed. As the head rotates, the centrifugal force created by its rotation expels the material through spaces between the heating elements. The heated feedstock forms discrete, generally spherical particles as it exists. The spherical microparticles so formed are then cooled by convection as they fall to the bottom of a collection chamber. The product is then collected and stored in suitable product containers.

The production of the spherical microparticles comprising the composition of the subject invention may be optimized by the use of a V-groove insert inside the spinner head. The insert is described in U.S. Pat. No. 5,851,454. The insert has grooves therein, which grooves have a uniform depth and width throughout their length so that highly uniform discrete spherical microparticles or other particles are produced. Using this or a similar insert, the spinning head is operated from about 50 Hz to about 75 Hz, from about 10% to about 40% power at temperatures, which yield liquiflash conditions.

Careful selection of the types and levels of excipient(s) control microparticle properties such as sphericity, surface morphology, and dissolution rate. The advantage of the process described above is that the microparticles are produced and collected from a dry feedstock without the use of any organic solvents.

The microparticles can also be prepared using other techniques such as fluid bed processes, extrusion/spheronization, spray/melt congealing or melt extrusion; however, the CEFORM™ process is the preferred method of manufacturing.

In an embodiment of the invention, it is preferred that the microparticles comprising the rapid absorption composition of the subject invention be coated with at least one coating after the spheronization process to mask the taste of the unpleasant tasting triptan in the microparticles. Useful coating formulations contain combinations of hydrophobic and hydrophilic polymers and optionally contain other excipient(s) conventionally employed in such coatings.

Useful hydrophobic polymers include (meth)acrylate/cellulosic polymers. Ethylcellulose (EC), hydroxypropylcellulose (HPC), hydroxypropylmethylcellulose (HPMC), and polymethacrylate polymers, such as Eudragit RS, Eudragit RL, E 100, and NE30D or mixtures thereof are useful. The preferred hydrophobic polymer is Ethylcellulose E45. The preferred hydrophilic polymer is Povidone K30.

The cellulosic coatings are generally applied to the microparticles after spheronization from an organic solvent solution(s). Typical solvents include one or more of acetone, alkyl alcohols (e.g., isopropyl alcohol), and the like. Coating devices used to coat the microparticles comprising the rapid absorption composition of the invention include those conventionally used in pharmaceutical processing. Fluidized bed coating devices are preferred. The coatings applied to the microparticles may contain ingredients other than the cellulosics. Thus, one or more colorants, flavorants, sweeteners, can also be used in the coating formulations.

Colorants used include food, drug and cosmetic colors (FD&C), drug and cosmetic colors (D&C) or external drug and cosmetic colors (Ext. D&C). These colors are dyes, lakes, and certain natural and derived colorants. Useful lakes include dyes absorbed on aluminum hydroxide or other suitable carriers.

Flavorants may be chosen from natural and synthetic flavoring liquids. An illustrative list of such agents includes volatile oils, synthetic flavor oils, flavoring aromatics, oils, liquids, oleoresins and extracts derived from plants, leaves, flowers, fruits, stems and combinations thereof. A non-limiting representative list of these includes citric oils, such as lemon, orange, grape, lime and grapefruit, and fruit essences, including apple, pear, peach, grape, strawberry, raspberry, cherry, plum, pineapple, apricot, or other fruit flavors.

Other useful flavorings include aldehydes and esters, such as benzaldehyde (cherry, almond); citral, i.e., alpha-citral (lemon, lime); neral, i.e., beta-citral (lemon, lime); decanal (orange, lemon); aldehyde C-8 (citrus fruits); aldehyde C-9 (citrus fruits); aldehyde C-12 (citrus fruits); tolyl aldehyde (cherry, almond); 2,6-dimethyloctanal (green fruit); 2-dodenal (citrus mandarin); mixtures thereof and the like.

Sweeteners may be chosen from the following non-limiting list: glucose (corn syrup), dextrose, invert sugar, fructose, and mixtures thereof (when not used as a carrier); saccharin and its various salts, such as sodium salt; dipeptide sweeteners such as aspartame; dihydrochalcone compounds, glycyrrhizin; Steva Rebaudiana (Stevioside); chloro derivatives of sucrose such as sucralose; and sugar alcohols such as sorbitol, mannitol, xylitol, and the like. Also contemplated are hydrogenated starch hydrolysates and the synthetic sweeteners such as 3,6-dihydro-6-methyl-1-1-1,2,3-oxathiazin-4-1-2,2-dioxide, particularly the potassium salt (acesulfame-K), and sodium and calcium salts thereof. The sweeteners may be used alone or in any combination thereof.

The diameter of the uncoated and coated microparticles range from about 100 μm in diameter to about 600 μm in diameter, preferably from about 200 μm to about 300 μm and most preferably from about 200 μm to about 250 μm. Coating levels of about 0% to about 100% w/w are effective, preferably about 15% to about 30% w/w and most preferably about 20% w/w.

II. Dosage Forms

Due to the substantially spherical nature of the coated and uncoated microparticles of the invention and their robustness, attributed to the high quantity of spheronization aid(s), the microparticles comprising the rapid absorption composition of the invention can be used in a number of different delivery systems. It is preferred that the microparticles comprising the rapid absorption composition of the invention are compressed into tablets with or without a cushioning matrix. Preferably, the microparticles are compressed into tablets without a cushioning matrix. However, the microparticles can also be incorporated into capsules, buccal tablets, sachets, and the like.

Tablets are the most widely used dosage form. Major reasons of tablet popularity as a dosage form are simplicity, low cost, and speed of production. Other reasons include stability of drug product, convenience in packaging, shipping, and dispensing. To the patient or consumer, tablets offer convenience of administration, ease of accurate dosage, compactness, portability, blandness of taste, and ease of administration,

Tablets may be plain, film or sugar coated, bisected, embossed, and/or layered. Tablets can also be made in a variety of sizes, shapes and colors. Tablets may be swallowed, chewed, or dissolved in the buccal cavity or under the tongue. Tablets may also be dissolved in water for local or topical application. Sterile tablets are normally used for parenteral solutions and for implantation beneath the skin.

In addition to the microparticles comprising the rapid absorption composition of the invention, a series of excipients are normally included in a tablet. The role of the excipients is to ensure that the tabletting operation can run satisfactorily and to ensure that tablets of specified quality are prepared. Depending on the intended main function, excipients to be used in tablets are subcategorized into different groups. However, one excipient can affect the properties of the tablet in a series of ways, and many substances used in tablet formulations can thus be described as multifunctional. As mentioned above, the excipients can include diluents (or fillers), disintegrants, binders, glidants, lubricants, antiadherents, sorbents, flavourants, colourants, etc.

Diluents or fillers are added to increase the bulk weight of the blend resulting in a practical size for compression. The ideal diluent or filler should fulfill a series of requirements, such as: be chemically inert, be non-hygroscopic, be biocompatible, possess good biopharmaceutical properties (e.g. water soluble or hydrophilic), good technical properties (such as compactibility and dilution capacity), have an acceptable taste and be cheap. As a single substance cannot fulfill all these requirements, different substances have gained use as diluents or fillers in tablets.

Lactose is a common filler in tablets. It possesses a series of good filler properties, e.g. dissolves readily in water, has a pleasant taste, is non-hygroscopic and fairly non-reactive and shows good compactibility. Other sugars or sugar alcohols, such as glucose, sucrose, sorbitol and mannitol, have been used as alternative fillers to lactose, primarily in lozenges or chewable tablets because of their pleasant taste. Mannitol has a negative heat of solution and imparts a cooling sensation when sucked or chewed.

Apart from sugars, perhaps the most widely used fillers are celluloses in powder forms of different types. Celluloses are biocompatible, chemically inert, and have good tablet forming and disintegrating properties. They are therefore used also as dry binders and disintegrants in tablets. They are compatible with many drugs but, owing to their hygroscopicity, may be incompatible with drugs prone to hydrolyse in the solid state. The most common type of cellulose powder used in tablet formulation is microcrystalline cellulose.

Another important example of a diluent or filler is dibasic and tribasic calcium phosphate, which is insoluble in water and non-hygroscopic but is hydrophilic, i.e. easily wetted by water. Other examples of diluents include but are not limited to di- and tri-basic starch, calcium carbonate, calcium sulfate, and modified starches. Many diluents are marketed in “direct compression” form, which adds other desirable properties, such as flow and binding. There are no typical ranges used for the diluents, as targeted dose and size of a tablet are variables that influence the amount of diluent that should be used.

A disintegrant may be included in the formulation to ensure that the tablet when in contact with a liquid breaks up into small fragments containing the microparticles comprising the rapid absorption composition of the invention, thereby obtaining the largest possible effective surface area for promoting rapid drug dissolution. The incorporation of disintegrants is especially important for immediate release products where rapid release of drug substance is required. Some disintegrants also function by producing gas, normally carbon dioxide, when in contact with a liquid. Such disintegrants are used in effervescent tablets and normally not in tablets that could be swallowed as a solid. The liberation of carbon dioxide is obtained by the decomposition of bicarbonate and carbonate salts in contact with an acidic liquid. The acidic pH is accomplished by the incorporation of a weak acid in the formulation. Examples of such acids include but are not limited to citric, tartaric, malic, fumaric, adipic, succinic and acid salts and anhydrides thereof. Acid salts may also include sodium dihydrogen phosphate, disodium dihydrogen pyrophosphate, acid citrate salts and sodium acid sulfite. While the food acids can be those indicated above, acid anhydrides of the above-described acids may also be used. Carbonate sources include dry solid carbonate and bicarbonate salts such as sodium bicarbonate, sodium carbonate, potassium bicarbonate and potassium carbonate, magnesium carbonate and sodium sesquicarbonate, sodium glycine carbonate, L-lysine carbonate, arginine carbonate and amorphous calcium carbonate. Mixtures of various acids and carbonate sources, as well as other sources of effervescence, can be used.

In direct compression tablets or encapsulation, a disintegrant(s) can be added to the excipient powder blend together with the microparticles comprising the rapid absorption composition of the invention prior to direct compression or encapsulation. Disintegrant(s) can also be used with products that are wet granulated. In wet granulation formulations, the disintegrant(s) is normally effective when incorporated into the microparticle (intragranularly). However, it may be more effective if added 50% intragranularly, and 50% extra-granularly (i.e., in the excipient powder blend). As mentioned above, excipients are often multifunctional. Thus, the diluent microcrystalline cellulose can also serve as a disintegrant. However, there are more effective agents referred to as superdisintegrants. It is preferred that the superdisintegrants have an Eq. moisture content at 25 C./90% RH of over 50%. A list of exemplary disintegrants, super disintegrants and other compounds with some disintegrant qualities are provided below:

| | | | | | Eq. Moisture | |
|---|---|---|---|---|---|---|
| Brand | | | Functional | | content at | |
| name | Common name | Classification | Category | Properties | 25 C./90% RH | Typical uses |
| CL- | Crospovidone | Polyvinylpoly | Tablet | Hygroscopic | 62% | Disintegrant |
| Kollidon | | pyrrolidone | super | Swelling-18% | | in dry and |
| | | | disintegrant | in 10 s, 45% | | wet |
| | | | | in 20 s | | granulation |
| Ac-Disol | Croscarmellose | Cellulose, | Tablet and | Hygroscopic | 88% | Disintegrant |
| Primellose | sodium | carboxymethyl | capsule | Wicking and | | for |
| | | ether, | super | swelling- | | capsules, |
| | | sodium salt, | disintegrant | 12% in 10 s, | | tablets and |
| | | crosslinked | | 23% in 20 s | | granules |
| Explotab | Sodium starch | Sodium | Tablet and | Swelling | | Disintegrant |
| Primojel | glycolate | carboxymethyl | capsule | capacity: in | | in dry and |
| | | starch | super | water swells | | wet |
| | | | disintegrant | up to 300 | | granulation |
| | | | | times its | | |
| | | | | volume | | |
| Explotab | Sodium starch | (Cross linked | Super | Swells to | | Disintegration |
| V17 | glycolate | low | disintegrant | greater | | and |
| | | substituted | | extent than | | dissolution |
| | | carboxymethyl | | explotab | | aid. Not for |
| | | ether)Sodium | | | | use in wet |
| | | carboxymethyl | | | | granulation |
| | | starch | | | | |
| Explotab | Sodium starch | (Cross linked | Super | | | Designed for |
| CLV | glycolate | low | disintegrant | | | wet |
| | | substituted | | | | granulation |
| | | carboxymethyl | | | | that utilize |
| | | ether)Sodium | | | | high shear |
| | | carboxymethyl | | | | equipment |
| | | starch, | | | | |
| | | highly cross | | | | |
| | | linked | | | | |
| L-HPC | Hydroxypropyl | Cellulose, 2- | Tablet and | Hygroscopic | 37% | Tablet |
| | cellulose, | hydroxypropyl | capsule | Swelling- | | disintegrant, |
| | low - | ether (low | disintegrant, | 13% in 10 s, | | binder in |
| | substituted | substituted) | tablet | 50% in 20 s | | wet |
| | | | binder | | | granulation |
| Amberlite | Polacrilin | Cation | Tablet | Swelling | | Tablet |
| IRP 88 | Potassium | exchange | disintegrant | ability | | disintegrant |
| | | resin | | | | |
| Starch | Starch, | Pregelatinized | Tablet and | Hygroscopic | 22% | Capsule and |
| 1500 | pregelatinized | starch | capsule | | | tablet |
| | | | diluent, | | | binder, |
| | | | disintegrant, | | | diluent, |
| | | | tablet | | | disintegrant |
| | | | binder | | | |
| Avicel | Microcrystalline | Cellulose | Tablet and | Hygroscopic | 18% | Binder/diluent- |
| | cellulose | | capsule | Swelling- | | has also |
| | | | diluent, | 12% in 10 s, | | some |
| | | | tablet | 18% in 20 s | | lubricant |
| | | | disintegrant | | | and |
| | | | | | | disintegrant |
| | | | | | | properties |

Binders (also sometimes called adhesives) are added to ensure that tablets can be formed with the required mechanical strength. Binders can be added in different ways: (1) As a dry powder, which is mixed with other ingredients before wet agglomeration; (2) As a solution, which is used as agglomeration liquid during wet agglomeration. Such binders are often referred to as “solution binders”, and (3) As a dry powder, which is mixed with the other ingredients before compaction (slugging or tabletting). Such binders are often referred to as “dry binders”. Common traditional solution binders are starch, sucrose, and gelatin. More commonly used binders with improved adhesive properties, are polymers such as polyvinylpyrrolidone and cellulose derivates such as for example hydropropyl methylcellulose. Examples of dry binders include microcrystalline cellulose and crosslinked polyvinylpyrrolidone. Other examples of binders include but are not limited to pregelatinized starches, methylcellulose, sodium carboxymethylcellulose, ethylcellulose, polyacrylamides, polyvinyloxoazolidone and polyvinylalcohols. Binders, if present, range in amounts from about greater than about 0% to about 25% depending on the binder used.

Glidants improve the flowability of the excipient powder by reducing intraparticulate friction. This is especially important during tablet production at high production speeds and during direct compaction. Examples of glidants include but are not limited to starch, talc, lactose, stearates (such as for example magnesium stearate), dibasic calcium phosphate, magnesium carbonate, magnesium oxide, calcium silicate, Cabosil™, colloidal silica (Syloid™) and silicon dioxide aerogels. Glidants, if present, range in amounts from greater than about 0% to about 20%, with amounts of about 0.1% to about 5% being typical.

Lubricants ensure that tablet formation and ejection can occur with low friction between the solid and the die wall. High friction during tabletting can cause a series of problems, including inadequate tablet quality (capping or even fragmentation of tablets during ejection, and vertical scratches on tablet edges) and may even stop production. Lubricants are thus included in almost all tablet formulations. Such lubricants include but are not limited to adipic acid, magnesium stearate, calcium stearate, zinc stearate, hydrogenated vegetable oils, sodium chloride, sterotex, polyoxyethylene, glyceryl monostearate, talc, polyethylene glycol, sodium benzoate, sodium lauryl sulfate, magnesium lauryl sulfate, sodium stearyl fumarate, light mineral oil and the like may be employed, with sodium stearyl fumarate being preferred. Waxy fatty acid esters, such as glyceryl behenate, sold as “Compritol™” products, can be used. Other useful commercial lubricants include “Stear-O-Wet™” and “Myvatex™ TL”. Mixtures are operable. Lubricants are used in amounts typically ranging from greater than about 0% to about 10%, with about 0.01% to about 5.0% by weight of the tablet preferred.

It is well known in the art that besides reducing friction, lubricants may cause undesirable changes in the properties of a tablet. The presence of a lubricant in the excipient powder is thought to interfere in a deleterious way with the bonding between the particles during compaction and thus reduce tablet strength. Because many lubricants are hydrophobic, tablet disintegration and dissolution are often retarded by the addition of a lubricant. Such negative effects are strongly related to the amount of lubricant present. Other considerations known in the art include the manner in which a lubricant is mixed, the total mixing time and the mixing intensity. In order to avoid these negative effects, hydrophilic substances may be substituted for the hydrophobic lubricants. Examples include, but are not limited to, surface-active agents and polyethylene glycol. A combination of hydrophilic and hydrophobic substances can also be used.

Anti-adherents reduce adhesion between the excipient powder mixture and the punch faces and thus prevent particles sticking to the punches, a phenomenon know in the art as “sticking” or “picking”, and is affected by the moisture content of the powder. One example of antiadherent is microcrystalline cellulose. Many lubricants such as magnesium stearate have also antiadherent properties. However, other substances with limited ability to reduce friction can also act as antiadherents. Such substances include for example talc and starch. Mixtures are operable. Antiadherents, if present, range from about 0% to about 20% by weight of the tablet depending on the antiadherent being used.

Sorbents are substances that are capable of sorbing some quantities of fluids in an apparently dry state. Thus, oils or oil-drug solutions can be incorporated into a powder mixture, which is granulated and compacted into tablets. Other examples of sorbing substances include microcrystalline cellulose and silica.

Flavouring agents are incorporated into a formulation to give the tablet a more pleasant taste or to mask an unpleasant one. The latter can also be achieved as described above by coating the tablet or the microparticles comprising the rapid absorption composition of the invention. Examples of flavouring agents include, but are not limited to, the flavouring agents described above for coating the microparticles comprising the rapid absorption composition of the invention.

If necessary, additional sweeteners, dyes and fragrances may be added to the tablet in addition to those already present in the coated microparticles comprising the rapid absorption composition of the invention. Such agents may be chosen from the non-limiting lists described above.

III. Directly Compressible Non-Cushioning Matrix Fast-Dispersing Oral Tablets.

In one embodiment, coated taste-masked microparticles comprising the rapid absorption composition of the invention are incorporated into fast-dispersing direct compression non-cushioning matrix oral tablets capable of dissolving in the mouth in less than about 40 seconds without the need for a conventional superdisintegrant and having a friability of less than about 1%. The fast-dispersing direct compression non-cushioning matrix oral tablet is comprised of the microparticles comprising the rapid absorption composition of the invention and a non-cushioning excipient mass comprising a linear polyol and/or lactose or maltose sugars, and optionally an inorganic salt, a cellulose or a cellulose derivative, or a mixture thereof. Applicants recently found that a robust fast-dispersing tablet could be produced using microparticles manufactured in accordance with the CEFORM™ technology, in the presence of lactose and/or linear polyol, and optionally microcrystalline cellulose (Avicel (MCC)) and/or an inorganic salt. The MCC in particular has been found to increase the robustness without a loss of disintegration behavior as one might expect from its high binding potential.

The linear polyol, lactose or maltose sugars, inorganic salt, cellulose or cellulose derivative may include a variety of directly compressible grades. No specific grade of these materials is precluded from use. In addition, some of these materials are offered in combination with other excipients as a co-blend or a co-processed material. Such co-blends or co-processed material are not precluded from use.

Typical linear polyols include powdered forms of mannitol, sorbitol, xylitol and directly compressible forms of mannitol, sorbitol and xylitol. The directly compressible grades of the linear polyols are preferred over the powdered forms. Mixtures are operable. The least preferred polyol is sorbitol with xylitol being the preferred polyol and mannitol being the most preferred linear polyol. The linear polyols are present in an amount from about greater than 0% to about 85%, preferably from about 20% to about 60% and most preferably from about 40% to about 50% by weight of the fast-dispersing direct compression non-cushioning matrix tablet. If lactose or maltose sugars are present, they may be present in an amount ranging from about 0% to about 85%, preferably from about 20% to about 60% and more preferably from about 40% to about 50% by weight of the fast-dispersing direct compression non-cushioning matrix tablet.

Typical non-limiting examples of inorganic salts include powdered forms of calcium carbonate, dibasic anhydrous calcium phosphate, dibasic dihydrate calcium phosphate, tribasic calcium phosphate, dihydrate calcium sulfate, monobasic sodium phosphate, dibasic sodium phosphate, anhydrous magnesium carbonate, alkaline diluent magnesium oxide and directly compressible grades of calcium carbonate (Destab®, Barcroft®, Cal-Carb®, Millicarb®, Sturcal®), directly compressible grades of dibasic anhydrous calcium phosphate (Anhydrous Emcompress®, A-Tab®, Di-Cafos® AN), directly compressible grades of dibasic calcium phosphate dihydrate (Emcompress®, Di-Tab®, Calstar®, Di-Cafos®), directly compressible grades of tribasic calcium phosphate (Tri-Cal®, Tri-Cafos®, Tri-Tab®), directly compressible grades of calcium sulfate (Compactrol®), directly compressible grades of anhydrous magnesium carbonate, directly compressible grades of magnesium aluminum silicate NF, and directly compressible grades of alkaline magnesium oxide (Destab®, Magnyox®). Mixtures are operable. It is preferred that the directly compressible grades of the inorganic salts be used, with the directly compressible grades of dibasic anhydrous calcium phosphate being the preferred directly compressible inorganic salt. The directly compressible inorganic salt comprising the excipient mass may be present in an amount ranging from about 0% to about 50%, preferably from about 5% to about 30% and most preferably from about 7.5% to about 15% by weight of the fast-dispersing direct compression non-cushioning matrix tablet.

Typical non-limiting examples of celluloses or directly compressible celluloses include powdered cellulose, (Cepo®, Elcema®, Sanacel®, Solka-Floc®), silicified microcrystalline cellulose (Prosolv®) and microcrystalline cellulose (Avicel®, Comprecel®, Emcocel®, Fibrocel®, Tabulose®, Vivacel®, Vivapur®). Microcrystalline cellulose is the preferred directly compressible cellulose. The directly compressible celluloses may be present in an amount ranging from about 0% to about 40%, preferably from about 5% to about 30% and most preferably from about 10% to about 20% by weight of the fast-dispersing direct compression non-cushioning matrix tablet.

Preferably, the microparticles comprising the rapid absorption composition of the invention and the non-cushioning matrix is combined in proportions such that the selective 5-HT agonist remains substantially within the microparticles when the microparticles and the non-cushioning matrix is compressed to obtain a fast-dispersing direct compression non-cushioning matrix oral tablet.

Although the microparticles to be used in the fast-dispersing direct compression non-cushioning matrix oral tablets may be uncoated, it is preferable that the microparticles be coated with at least one taste-masking coating to mask the taste of any unpleasant selective 5-HT agonist comprising the rapid absorption composition of the invention. Useful coating formulations contain polymeric ingredients as well as excipient(s) conventionally employed in such coatings and can be chosen from the non-limiting lists described above.

The fast-dispersing direct compression non-cushioning matrix oral tablets comprising the microparticles and the excipient mass may further comprise a disintegrant not having superdisintegrant properties to aid in the disintegration of the tablet and hence the dissolution of the selective 5-HT agonist from within the microparticles. Such disintegrants may be chosen from the non-limiting list described above and may me present in an amount from about 0% to about 40%, preferably from about 5% to about 30% and most preferably from about 10% to about 20% by weight of the fast-dispersing direct compression non-cushioning matrix tablet.

The fast-dispersing direct compression non-cushioning matrix oral tablets typically have a hardness in the range of from about 4 N to about 60 N, preferably from about 15 N to about 35 N and most preferably from about 20 N to about 30 N. The friability of such tablets typically range from about 0% to about 10%, preferably from about 0.1% to about 2.0% and most preferably from about 0.4% to about 0.8%.

In a preferred embodiment, the microparticles comprising the rapid absorption composition of the invention are incorporated into fast-dispersing direct compression non-cushioning matrix oral tablets capable of dissolving in the mouth in less than 30 seconds and having a friability of less than 1%. It is preferred that the microparticles be coated with at least one taste-masking coat. The non-cushioning matrix comprises a linear polyol, a superdisintegrant in an amount less than about 2.5% by weight of the tablet and optionally an inorganic salt, a cellulose, or a cellulose derivative. The linear polyol and optionally the inorganic salt, cellulose or cellulose derivatives may be chosen from the non-limiting lists described above. The preferred linear polyol is directly compressible mannitol, with microcrystalline cellulose and directly compressible dibasic calcium phosphate dihydrate being the preferred cellulose and inorganic salt respectively. The preferred superdisintegrant is Kollidon CL. Superdisintegrants are present in an amount ranging from about 0% to about 3%, preferably from about 2% to about 3% and most preferably from about 2.5% to about 3% by weight of the tablet.

IV. Directly Compressible Cushioning Matrix Fast-Dispersing Oral Tablets.

The microparticles comprising the rapid absorption composition of the invention may also be incorporated into fast-dispersing oral tablets with a cushioning matrix. The preferred cushioning matrix is a processed excipient of a floss type substance of mixed polysaccharides converted into amorphous fibers.

The preparation of floss type cushioning matrices suitable for use in the present invention is disclosed in U.S. Pat. Nos. 5,622,719, 5,851,553, 5,866,163 all for “Process and Apparatus for Making Rapidly Dissolving Dosage Units and Product Therefrom” and U.S. Pat. No. 5,895,664 for “Process for forming quickly dispersing comestible unit and product therefrom”, the contents of which are incorporated herein by reference. Preferably, the floss type cushioning matrix is a “shearform matrix” produced by subjecting a feedstock which contains a sugar carrier to flash-heat processing.

In the flash-heat process, the feedstock is simultaneously subjected to centrifugal force and to a temperature gradient, which raises the temperature of the mass to create an internal flow condition, which permits part of it to move with respect to the rest of the mass. The flowing mass exits through openings provided in the perimeter of a spinning head. The temperature gradient is supplied using heaters or other means which cause the mass' temperature to rise. Centrifugal force in the spinning head flings the internally flowing mass outwardly, so that it reforms as discrete fibers with changed structures.

An apparatus, which produces suitable conditions, is a modified floss-making machine, such as that described in U.S. Pat. No. 5,834,033, entitled “Apparatus for Melt Spinning Feedstock Material having a Flow Restricting Ring”. The entire content of that application is hereby incorporated by reference.

Typically, spinning is conducted at temperatures and speeds of about 180° C. to 250° C. and 3,000 to 4,000 rpm, respectively.

A suitable spinner head is disclosed for example in U.S. Pat. No. 5,458,823, which contents is hereby incorporated by reference. However, other useful apparatuses or processes that provide similar forces and temperature gradient conditions can be used.

The cushioning matrix or floss particles can be chopped using the apparatus discussed in U.S. Pat. No. 5,637,326. Any other device having a similar function is also suitable.

The shearform matrix used herein includes a carrier, or feedstock material, which comprises at least one material selected from materials which are capable of undergoing the physical and/or chemical changes associated with flash heat processing. Useful carriers include carbohydrates, which become free-form particulates when flash heat processed. Saccharide-based carriers, including saccharides (i.e., sugars), polysaccharides and mixtures thereof can be used.

The feedstocks used in the invention can include carriers chosen from various classes of “sugars”. “Sugars” are those substances, which are based on simple crystalline mono- and di-saccharide structures, i.e., based on C₅ and C₆ sugar structures. The sugars can include glucose, sucrose, fructose, lactose, maltose, pentose, arabinose, xylose, ribose, mannose, galactose, sorbose, dextrose and sugar alcohols, such as sorbitol, mannitol, xylitol, maltitol, isomalt, sucralose and the like and mixtures thereof. Sucrose is the preferred sugar.

Useful mixtures of carriers include the sugars listed above along with additional mono- di-, tri- and polysaccharides. Additional saccharides can be used in amounts of up to 50% by weight of the total sugar, preferably up to 30%, and most preferably up to 20%.

Optionally, the polysaccharides can be used alone as carriers. Polysaccharide carriers include polydextrose and the like. Polydextrose is a non-sucrose, essentially non-nutritive, carbohydrate substitute. It can be prepared through polymerization of glucose in the presence of polycarboxylic acid catalysts and polyols. Generally, polydextrose is commercially available in three forms: polydextrose A and polydextrose K, which are powdered solids; and polydextrose N, which is supplied as a 70% solution. U.S. Pat. No. 5,501,858 discusses polydextrose, the content of which is incorporated herein by reference.

If other carrier materials are used, they are employed in combination with sugar and not as a total replacement therefor. For example, maltodextrins may be employed. Maltodextrins include mixtures of carbohydrates resulting from the hydrolysis of a saccharide. They are solids having a dextrose equivalent (DE) of up to and including 65.

The carrier can also include malto-oligo-saccharides produced by selective hydrolysis of cornstarch. A general description of malto-oligo-saccharides useful herein is set forth in U.S. Pat. Nos. 5,347,341 and 5,429,836, which contents are incorporated herein by reference.

If cushioning matrix systems are to be used, the following two systems, which are devoid of glycerine, are preferred.

In the first system, xylitol is added to a mixture of saccharide-based carrier and one or more additional sugar alcohols, with sorbitol being favored as an additional sugar alcohol. The carrier mix is flash-heat processed to provide a shearform floss-cushioning matrix having self-binding properties. Shearform flosses made using sucrose, sorbitol and xylitol have been found to yield particularly effective self-binding properties. They exemplify “single floss” or “unifloss” systems.

The second system makes separate xylitol-containing binder flosses. The binder flosses (“binder portions”) are combined with base flosses (“base portions”), which contain a different sugar alcohol and a saccharide. Preferably, the base floss contains sorbitol and sucrose, while the binder floss contains xylitol. These are termed “dual floss” systems.

The ingredients, which increase cohesiveness and give self-binding properties, preferably include sugar alcohols, such as sorbitol, xylitol, maltitol, mannitol and mixtures thereof, all of which form flosses. Other sugar alcohols, especially hygroscopic ones, are contemplated.

Xylitol and sorbitol are the preferred sugar alcohols. Effective amounts of xylitol in the flosses are between about 0.5% and 25%, and preferably about 10% by weight of the floss. Sorbitol is used in the flosses in amounts of about 0.5% to about 40% by weigh of the floss.

When sorbitol and xylitol are used, the ratio of sorbitol to xylitol is from about 1:0.1 to about 1:10.

In dual floss systems, about 20% to about 80%, preferably about 34%, of the total floss content is xylitol-containing, or binder, floss. Likewise, the sorbitol containing, or base, floss may be about 20% to about 80% of the total floss. In some “dual floss” embodiments, xylitol-containing flosses are first mixed with active ingredient(s), and then mixed with sucrose/sorbitol flosses.

Regardless of the number of flosses, the total floss content preferably includes about 50% to about 85% sucrose, about 5% to about 20% sorbitol and about 5% to about 25% xylitol.

In some cases, flosses are used along with bio-affecting, or active, microspheres in the tabletting process. Often, xylitol-containing floss is added to microspheres of one or more active agents first and then non-xylitol-containing floss is added. Typically, the weight ratio of total floss to microspheres is about 1:1. In these instances, about 5% to about 25% of the floss is xylitol.

Whereas prior art floss type matrices conventionally included a liquid binding additive such as glycerine, the floss type matrices described herein do not. Instead, they get their enhanced cohesiveness, self-binding character and flowability directly from the matrix or feedstock ingredients and the processing used.

The amorphous shearform matrix of the present invention is preferably made from a feedstock, which includes sucrose, sorbitol, and xylitol. As set forth in U.S. Pat. No. 5,869,098, entitled “Fast Dissolving Comestible Units Formed under High Speed/High Pressure Conditions”, these compositions promote recrystallization and tabletting of the matrix-containing mixes to a level sufficient to provide particulate flowability for use in high speed and high pressure tabletting equipment.

The rapid absorption compositions to be processed into comestible units, or tablets, can contain conventional excipients. Conventional quantities of these excipients may be incorporated into one or more of the matrices or may be mixed therewith prior to tabletting. Useful amounts of conventional excipients range from about 0.01% to about 80% by weight, based on the weight of the cushioning matrices or formulations in which they are used. The quantities may vary from these amounts, depending on the functions of the excipients and the characteristics desired in the matrices and/or the final tablet compositions.

Conventional tabletting excipients may be selected from the non-limiting lists described above.

The preformed matrices produced in accordance herewith may be rendered more crystalline by one or more of the following crystallizing techniques. The nature of the shearform matrix feedstock determines whether the matrix is re-crystallized after it is formed. Nonetheless, “crystallization” and “recrystallization” are used interchangeably herein.

One technique for recrystallizing involves the use of crystallization enhancers. These are used after the shearform floss has been formed, but before the shearform floss-containing composition is tableted. Suitable crystallization enhancers include ethanol, polyvinyl-pyrrolidone, water (e.g. moisture), glycerine, radiant energy (e.g., microwaves) and the like, with combinations being useful. When they are physical materials, typical amounts of these enhancers range from about 0.01% to about 10.0% by weight of the tablet composition.

Another technique relates to the use of crystallization modifiers. These crystallization modifiers are floss ingredients, used at levels of about 0.01% to about 20.0% by weight of the floss.

Surfactants are preferred crystallization modifiers. Other materials, which are non-saccharide hydrophilic organic materials may also be used. Useful modifiers preferably have a hydrophilic to lipid balance (HLB) of about 6 or more. Such materials include, without limitation, anionic, cationic, and zwitterionic surfactants as well as neutral materials with suitable HLB values. Hydrophilic materials having polyethylene oxide linkages are effective. Those with molecular weights of at least about 200, preferably at least 400, are highly useful.

Crystallization modifiers useful herein include: lecithin, polyethylene glycol (PEG), propylene glycol (PPG), dextrose, the SPANS® and TWEENS® which are commercially available from ICI America, and the surface active agents known as “Carbowax®”. Generally, the polyoxyethylene sorbitan fatty acid esters called TWEEN®s, or combinations of such modifiers are used. Crystallization modifiers are usually incorporated into matrices in amounts of between about 0% and 10%.

Optionally, the shearform matrices are allowed to re-crystallize, with or without added crystallization modifiers, either before or after they are combined with the non-matrix component(s), e.g., the bio-affecting additive(s). When recrystallization occurs before tabletting, the recrystallization level of the matrix generally reaches at least about 10%. The use of such partially recrystallized matrices leads to compositions that are free flowing and tabletable using conventional machines. U.S. Pat. No. 5,597,416 describes a process for recrystallizing in the presence of excipients.

Methods for effecting the recrystallization of the shearform matrices include: use of Tween® 80 or other crystallization modifier(s) in the shearform matrix premix; aging of the shearform matrix for up to several weeks, contacting the shearform matrix with sufficient moisture and heat to induce crystallization, and treating the shearform matrix or the shearform floss-containing composition with ethanol or another crystallization enhancer. Mixtures are operable.

When a surfactant, such as a Tween® is used, about 0.001% to about 1.00% is included in the shearform matrix preblend as a crystallization modifier. Following preblending, the formulations are processed into flosses, then chopped and used, with or without excipients, to make tablets. Mixtures of surfactants can also be used.

Aging may be used to re-crystallize the shearform matrix or floss. The aging process involves a two-step process. First, the shearform matrix, which typically contains at least one crystallization modifier, is formed, chopped and allowed to stand in closed or sealed containers without fluidization or other agitation under ambient conditions, e.g., at room temperature and atmospheric pressure, for up to several days, preferably for about 1 to about 3 days. Later, the shearform matrix is mixed, and optionally further chopped, with one or more other ingredients. The mix is then aged by allowing it to stand for an additional period of about 1 to about 3 days. Generally, the two-step aging process takes a total of about one week, with periods of about 4 to about 5 days being typical.

The flosses may also be re-crystallized by subjecting them to increased heat and moisture. This process is similar to aging, but involves shorter periods of time. Using a fluidized bed apparatus or other suitable device, chopped floss is fluidized while heating, at ambient humidity and pressure, to temperatures of about 25° C. to about 50° C. Typically, the temperature is monitored to minimize clumping of floss particles during this operation. If any clumping occurs, the floss particles must be sieved before being further processed into tablets. Heating times of about 5 to about 30 minutes are typical.

When ethanol is used as a crystallization enhancer, it is used in amounts, based upon the weight of the shearform matrix, of about 0.1% to about 10%, with amounts of about 0.5% to about 8.0% being very effective. The preformed shearform matrix is contacted with ethanol. Excess ethanol is evaporated by drying for about an hour at about 85° F. to about 100° F., with 95° F. being highly useful. The drying step is carried out using tray drying, a jacketed mixer or other suitable method. Following ethanol treatment, the matrix becomes partially re-crystallized on standing for a period ranging from about a few hours up to several weeks. When the floss is about 10% to about 30% re-crystallized, it is tableted after blending with other ingredients. The tabletting compositions flow readily and are cohesive.

Re-crystallization of the matrix may take place in the presence of one or more bio-affecting agents or other excipients.

Re-crystallization of the matrix can be monitored by measuring the transmittance of polarized light therethrough or by the use of a scanning electron microscope. Amorphous floss or shearform matrix does not transmit polarized light and appears black in the light microscope when viewed with polarized light. Using bright field microscopy or the scanning electron microscope, the surface of the floss appears very smooth. In this condition, it is 0% re-crystallized. That is, the floss is 100% amorphous.

Re-crystallization of amorphous shearform matrix starts at the surface of the mass and can be modified, e.g., accelerated, by the presence of crystallization modifiers, as well as moisture. When TWEEN®s assist the re-crystallization, initiation of re-crystallization is evidenced by a birefringence observed on the surface of the shearform matrix (floss) as viewed with polarized light. There are faint points of light riddled throughout the matrix surface. When birefringence appears, re-crystallization has begun. At this stage, re-crystallization is between about 1% and about 5%.

As re-crystallization proceeds, the birefringence on the surface of the shearform matrix grows continually stronger and appears brighter. The points of light grow in size, number and intensity, seeming to almost connect. Using bright field or scanning electron microscopy, the surface of the shearform matrix appears wrinkled. At this point, about 5 to 10% recrystallization has occurred.

Surfactant (e.g., TWEEN® 80) droplets become entrapped within the matrix. These droplets are obscured as re-crystallization proceeds. As long as they are visible, the shearform matrix floss is generally not more than about 10% to about 20% re-crystallized. When they are no longer observable, the extent of re-crystallization is no more than about 50%.

The re-crystallization of the shearform matrix floss results in reduction of the total volume of material. Ordered assays of molecules take up less space than disordered arrays. Since re-crystallization begins at the surface of the shearform matrix floss, a crust is formed which maintains the size and shape of the shearform matrix floss. There is an increase in the total free volume space within the floss as re-crystallization nears completion, which manifests itself as a void inside the floss. This is evidenced by a darkened central cavity in light microscopy and a hollow interior in scanning electron microscopy. At this stage, the shearform matrix floss is believed to be about 50% to about 75% re-crystallized.

The intensity of transmitted polarized light increases as the shearform matrix floss becomes more crystalline. The polarized light can be measured by a photon detector and assigned a value against calculated standards on a gray-scale.

The final observable event in the recrystallization of the shearform matrix floss is the appearance of fine, “cat whisker-like” needles and tiny blades, which grow and project from the surface of the floss. These crystals, believed to be sorbitol (cat whiskers) and xylitol (blades), literally cover the floss like a blanket of fuzz. These features can be easily recognized by both light and electron microscopes. Their appearance indicates the final stage of recrystallization. The floss is now about 100% re-crystallized, i.e., substantially non-amorphous.

The matrix portions of the tabletable composition are typically formed via flash-heat processing into floss. The floss strands are macerated or chopped into rods for further processing. Rods of chopped floss have lengths of about 50 μm to about 500 μm.

Other ingredients, which may be included, are conventional tablet excipients. Additional fragrances, dyes, flavors, sweeteners (both artificial and natural) may also be included, if necessary even though the microspheres to be incorporated into the floss are already taste-masked. The additional excipients, which can be included, have been described above.

The following non-limiting examples illustrate the invention:

### EXAMPLE 1

Uncoated Microparticles (Low Macrogol Fatty Acid Ester Content):

The following rapid absorption formulation is prepared:

| Ingredients | Amount (%) |
|---|---|
| Sumatriptan Succinate | 30 |
| Glyceryl Palmitostearate^{a} | 65 |
| Macrogol Fatty Acid Ester^{b} | 5 |
| Total | 100 |

^{a}Precirol ® ato 5
^{b}Gelucire ® 50/13

Each of the ingredients is transferred into a Robot Coupe (10L bowl) in the following order:

1 . ½ of the glyceryl palmitostearate,

2. All of the sumatriptan succinate,

3. All of the macrogol fatty acid ester,

4. Remainder of the glyceryl palmitostearate.

The ingredients are blended at low shear (600rpm) for about 1 minute after which the speed is increased to 3000 rpm and further blended for about 4½ minutes.

The resulting blend was spheronized using the following process parameters (liquiflash conditions). The process called for a percent power input of about 22% and a head speed of about 55 Hz. The head used is a CEFORM® 3″ V-groove head. The process temperature at which the blend was exposed to during the spheronization is about 117° C. to about 118° C.

Samples of the microparticles were taken during the spheronization process to show uniformity. Dissolution profiles meet the guidelines recommended for an immediate release product.

In process samples were also taken during the screening step. All assay values are within target values and dissolution results are consistent. P.S.A. data report value but the D₅₀ is in the desired range of 200 μm-300 μm. The microparticle morphology was examined under a polarized light microscope and reported as spherical and uniform in shape. Thus, the microparticles were deemed acceptable for coating.

The dissolution profile of the microparticles was determined under the following dissolution conditions:

Medium: 900 mI, Dl water,

Method: USP Apparatus II at 60 rpm at 37° C.

The results are presented below as a % release of the total sumatriptan succinate in the microparticles:

| Time | Mean | Std. | Min. | Max. |
|---|---|---|---|---|
| (minutes) | (%) | Dev. (%) | (%) | (%) |
| 0 | 0 | 0 | 0 | 0 |
| 10 | 96 | 3 | 93 | 102 |
| 20 | 105 | 3 | 102 | 110 |
| 30 | 106 | 3 | 102 | 111 |
| 40 | 106 | 3 | 102 | 111 |
| 50 | 106 | 3 | 102 | 111 |
| 60 | 106 | 3 | 102 | 111 |

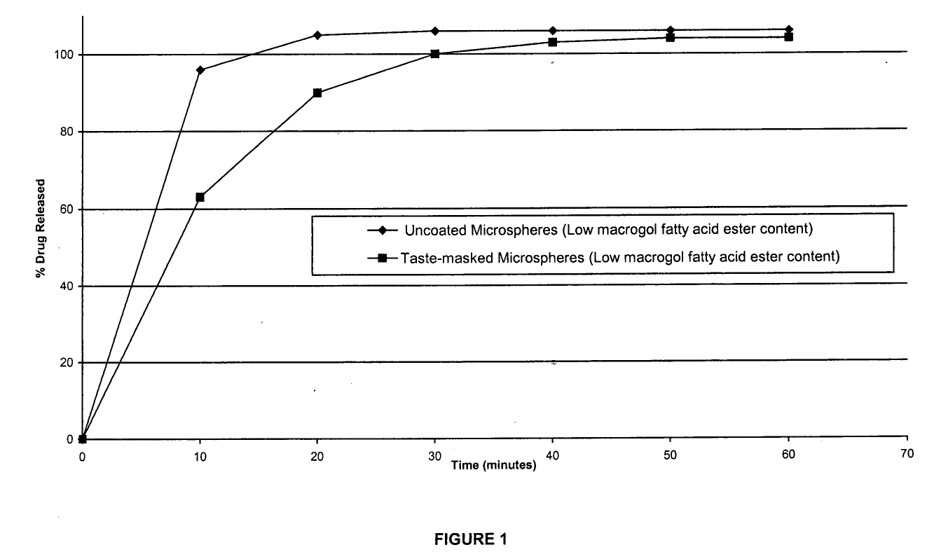

The dissolution profile of the above microparticles prepared as described above is shown in FIG. 1.

### EXAMPLE 2

Coated Microparticles (Low Macrogol Fatty Acid Ester Content):

The microparticles are produced according to the same manufacturing process described above in Example 1. The microparticles thus obtained are then coated for taste masking with a coating solution containing Ethocel E45 and Povidone K30 in a ratio of Ethocel E45:Povidone K30 of 7:3

The solution is prepared by placing a solvent mixture of acetone and IPA in a ratio of acetone:IPA of 6:4 in a container under an IKA Eurostar stirrer. The solvent is mixed for about 30 seconds before the 7:3 ratio of Ethocel E45:Povidone K30 is added to the vortex. Mixing is continued until the Ethocel E45 and Povidone K30 are completely dispersed (about 30 minutes).

Coating of the microparticles obtained from Example 1 is carried out in a Glatt GPCG-3 Wurster. The parameters are adjusted during the coating procedure to ensure adequate fluidization and minimize agglomeration. The process parameters are set as indicated below:

| | Units of | Initial |
|---|---|---|
| | Measurement | setting |
| Inlet air temperature | ° C. | 36 |
| Outlet air temperature | ° C. | 24-27 |
| Filter shake interval/duration | Seconds | 20 S/3 s |
| Atomization Air pressure | Bar | 2.3 |
| Exhaust Air Flap | — | 17.5% |
| Product Temperature | ° C. | 23-26 |

The coating process is continued until a target coating level of 20% w/w is achieved. At this point the coating process is terminated and the drying can commence.

The dissolution profile of the coated microparticles is determined under the same conditions as described for the uncoated microparticles in Example 1.

The results of the dissolution of the coated microparticles are presented below as a % release of the total sumatriptan succinate in the microparticles:

| Time | Mean | Std. | Min. | Max. |
|---|---|---|---|---|
| (minutes) | (%) | Dev. (%) | (%) | (%) |
| 0 | 0 | 0 | 0 | 0 |
| 10 | 63 | 3 | 60 | 68 |
| 20 | 90 | 3 | 87 | 95 |
| 30 | 100 | 2 | 98 | 104 |
| 40 | 103 | 2 | 101 | 107 |
| 50 | 104 | 2 | 102 | 108 |
| 60 | 104 | 2 | 102 | 108 |

The dissolution profile of the above-coated microparticles is shown in FIG. 1.

### EXAMPLE 3

Uncoated Microparticles (High Macrogol Fatty Acid Ester Content):

The following rapid absorption formulation is prepared:

| Ingredients | Amount (%) |
|---|---|
| Sumatriptan Succinate | 30 |
| Glyceryl Palmitostearate^{a} | 35 |
| Macrogol Fatty Acid Ester^{b} | 35 |
| Total | 100 |

^{a}Precirol ® ato 5
^{b}Gelucire ® 50/13

The ingredients are mixed and the spheronization process carried out as described in Example 1.

The dissolution profile of the microparticles was determined under the same conditions as set out in Example 1. The results are presented below as a % release of the total sumatriptan succinate in the microparticles:

| Time | Mean | Std. | Min. | Max. |
|---|---|---|---|---|
| (minutes) | (%) | Dev. (%) | (%) | (%) |
| 0 | 0 | 0 | 0 | 0 |
| 10 | 98 | 1 | 97 | 100 |
| 20 | 99 | 1 | 97 | 101 |
| 30 | 99 | 1 | 97 | 101 |
| 40 | 99 | 1 | 97 | 101 |
| 50 | 99 | 1 | 98 | 101 |
| 60 | 99 | 1 | 98 | 101 |

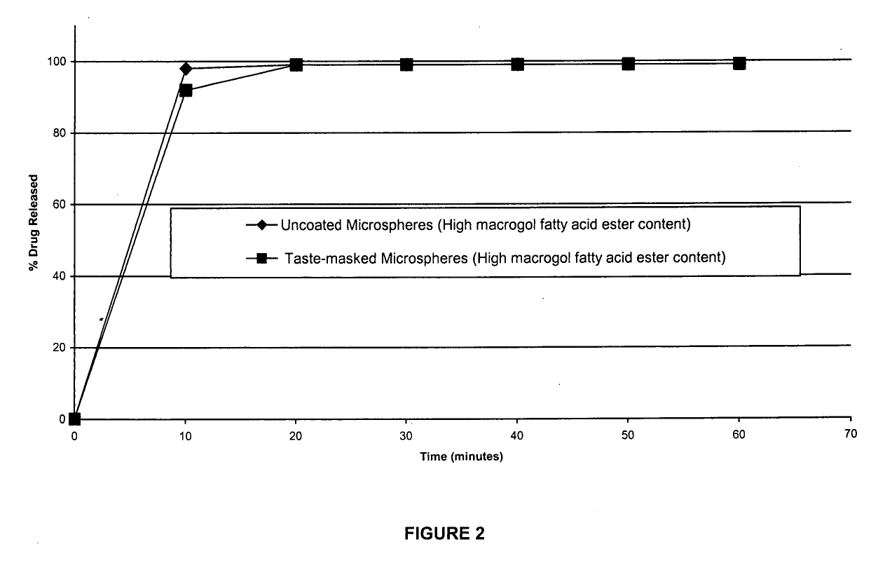

The dissolution profile of the microparticles is shown in FIG. 2.

### EXAMPLE 4

Coated Microparticles (High Macrogol Fatty Acid Ester Content):

Coating of the microparticles obtained in Example 3 is carried out as described in Example 2.

The dissolution profile of the microparticles was determined under the same conditions as set out in Example 1. The results are presented below as a % release of the total sumatriptan succinate in the microparticles:

| Time | Mean | Std. | Min. | Max. |
|---|---|---|---|---|
| (minutes) | (%) | Dev. (%) | (%) | (%) |
| 0 | 0 | 0 | 0 | 0 |
| 10 | 92 | 1 | 91 | 94 |
| 20 | 99 | 1 | 96 | 100 |
| 30 | 99 | 1 | 98 | 100 |
| 40 | 99 | 1 | 99 | 100 |
| 50 | 99 | 1 | 98 | 100 |
| 60 | 99 | 1 | 99 | 100 |

The dissolution profile of the coated microparticles is shown in FIG. 2.

### EXAMPLE 5

Fast-Dispersing Direct Compression Non-Cushioning Matrix Dosage Form (Low Macrogol Fatty Acid Ester Content):

The coated microparticles as prepared in Example 2 were used in the following tablet composition:

| | % w/w of 50 mg |
|---|---|
| Tablet Component | Tablet |
| Sumatriptan Succinate coated | 35.21 |
| microparticles (low macrogol | |
| fatty acid ester content) | |
| Mannitol^{a} | 44.64 |
| Microcrystalline Cellulose^{b} | 15.00 |
| Kollidon CL | 2.00 |
| Silicon dioxide^{c} | 0.50 |
| Sodium Stearyl Fumarate^{d} | 1.00 |
| Intense Peppermint Flavor | 0.75 |
| Acesulfame K | 0.60 |
| Magnasweet ® 100 | 0.30 |
| Total | 100.00 |

^{a}Pearlitol 400DC ®
^{b}Avicel ® PH101
^{c}Syloid ® 244FP
^{d}PRUV ®

Each of the components is transferred into a 4qt V-blender and blended in the order specified below:

1. ½ of the mannitol,

2. All of the coated sumatriptan microparticles,

3. Remainder of the mannitol.

The above mixture is blended for about 3 minutes with the intensifier bar on after which the following components are added:

4. All of the Acesulfame K,

5. All of the Magnasweet® 100

6. All of the microcrystalline cellulose,

7. All of the intense peppermint flavor.

The mixture is again blended for about 3 minutes with an intensifier bar after which the following component is added and mixed for about 2 minutes with the intensifier bar on:

8. All of the silicon dioxide,

The final components added are:

9. All of the Kollidon CL, and

10. All of the Sodium Stearyl Fumarate.

The mixture is now blended with the intensifier bar off for about 2 minutes. The blend subsequently compressed to a target weight of 800 mg in a Picola tablet press.

The tablets formed typically have a hardness value of about 23 N to about 27 N, a thickness of about 4.24 mm to about 4.26 mm and a friability of about less than 1%.

The dissolution profile of the tablet is determined under the following conditions:

Medium: 900 ml DI water,

Method: USP Apparatus II at 60 rpm at 37° C.

The fast-dispersing direct compression non-cushioning matrix tablet (low macrogol fatty acid ester) produced the following dissolution profile:

| Time | Mean | Std. | Min. | Max. |
|---|---|---|---|---|
| (minutes) | (%) | Dev. (%) | (%) | (%) |
| 0 | 0 | 0 | 0 | 0 |
| 10 | 88 | 7 | 75 | 95 |
| 20 | 102 | 3 | 100 | 107 |
| 30 | 103 | 2 | 101 | 108 |
| 40 | 104 | 3 | 101 | 109 |
| 50 | 104 | 3 | 101 | 109 |
| 60 | 104 | 3 | 101 | 109 |

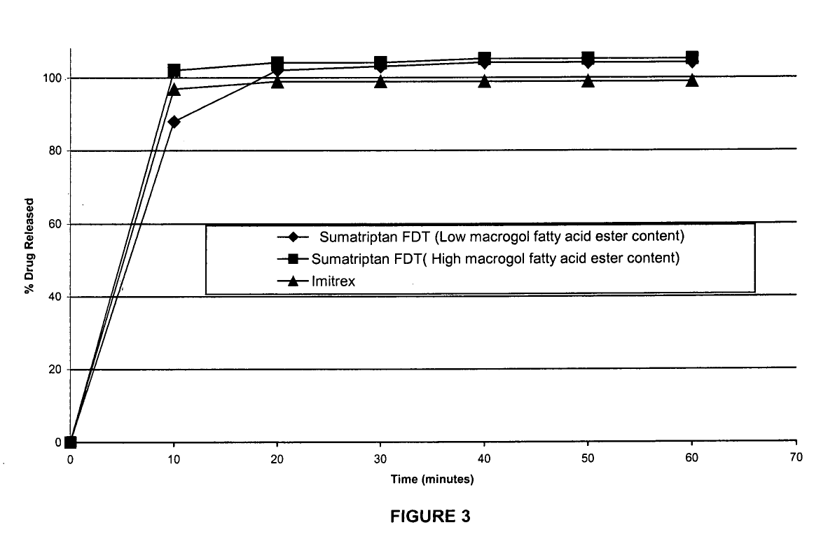

The dissolution profile of the above tablet is shown in FIG. 3.

### EXAMPLE 6

Fast-Dispersing Direct Compression Non-Cushioning Matrix Dosage Form (High Macrogol Fatty Acid Ester Content):

The coated microparticles as prepared in Example 4 were used in the following tablet composition:

| | % w/w of 50 mg |
|---|---|
| Tablet Component | Tablet |
| Sumatriptan Succinate coated | 35.21 |
| microparticles (high macrogol | |
| fatty acid ester content) | |
| Mannitol^{a} | 44.64 |
| Microcrystalline Cellulose^{b} | 15.0 |
| Kollidon CL | 2.0 |
| Silicon dioxide^{c} | 0.50 |
| Sodium Stearyl Fumarate^{d} | 1.00 |
| Intense Peppermint Flavor | 0.75 |
| Acesulfame K | 0.60 |
| Magnasweet ® 100 | 0.30 |
| Total | 100.00 |

^{a}Pearlitol 400DC ®
^{b}Avicel ® PH101
^{c}Syloid ® 244FP
^{d}PRUV ®

The tablet components are mixed and tableted as described in Example 5. The resulting tablets weigh about 800 mg each and typically have a hardness value of about 28 N to about 30 N, a thickness of about 4.19 mm to about 4.20 mm and a friability of about less than 1%.

The dissolution profile of the tablet is determined as described in Example 5. The fast-dispersing direct compression non-cushioning matrix tablet (high macrogol fatty acid ester content) produced the following dissolution profile:

| Time | Mean | Std. Dev. | Min. | Max. |
|---|---|---|---|---|
| (minutes) | (%) | (%) | (%) | (%) |
| 0 | 0 | 0 | 0 | 0 |
| 10 | 102 | 1 | 100 | 103 |
| 20 | 104 | 1 | 101 | 105 |
| 30 | 104 | 1 | 101 | 106 |
| 40 | 105 | 1 | 101 | 106 |
| 50 | 105 | 1 | 101 | 106 |
| 60 | 105 | 1 | 102 | 106 |

The dissolution profile of the above tablet is shown in FIG. 3.

### EXAMPLE 7

Uncoated Microparticles II (High Macrogol Fatty Acid Ester Content):

The following rapid absorption formulation is prepared:

| Ingredients | Amount (%) |
|---|---|
| Sumatriptan Succinate | 40 |
| Glyceryl Palmitostearate^{a} | 25 |
| Macrogol Fatty Acid Ester^{b} | 35 |
| Total | 100 |

^{a}Precirol ® ato 5
^{b}Gelucire ® 50/13

The ingredients are mixed and the spheronization process carried out as described in Example 1.

### EXAMPLE 8

Coated Microparticles II (High Macrogol Fatty Acid Ester Content):

Coating of the microparticles obtained in Example 7 is carried out as described in Example 2.

### EXAMPLE 9

Fast-Dispersing Direct Compression Non-Cushioning Matrix Dosage Form II (High Macrogol Fatty Acid Ester Content):

The coated microparticles as prepared in Example 8 were used and made as described in Example 6.

The dissolution profile of the tablet is determined as described in Example 5 produced the following dissolution profile:

| Time | Mean | Std. Dev. | Min. | Max. |
|---|---|---|---|---|
| (minutes) | (%) | (%) | (%) | (%) |
| 0 | 0 | 0 | 0 | 0 |
| 5 | 99 | 1 | 97 | 100 |
| 10 | 101 | 1 | 99 | 104 |
| 20 | 101 | 1 | 100 | 103 |
| 30 | 102 | 1 | 100 | 104 |
| 45 | 102 | 1 | 100 | 104 |
| 60 | 102 | 1 | 101 | 104 |

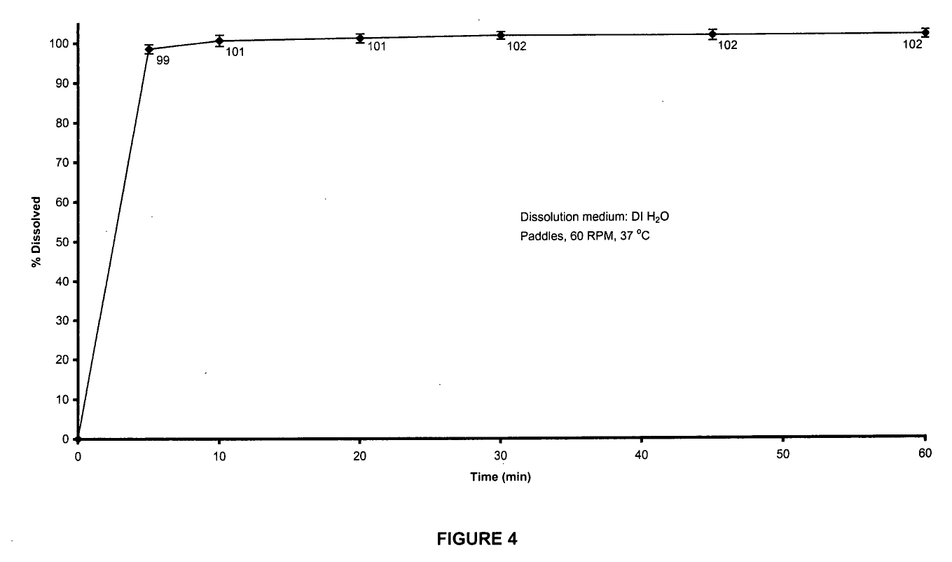

The dissolution profile of the tablet is shown in FIG. 4.

### EXAMPLE 10

Comparative Dissolution Profile of Prior Art 50 mg Imitrex® Tablet:

The dissolution of the prior art 50 mg Imitrex® Tablet was carried out under the same conditions described in Examples 5 and 6. The prior art 50 mg Imitrex® Tablet produced the following dissolution profile:

| Time | Mean | Std. Dev. | Min. | Max. |
|---|---|---|---|---|
| (minutes) | (%) | (%) | (%) | (%) |
| 0 | 0 | 0 | 0 | 0 |
| 10 | 97 | 2 | 94 | 99 |
| 20 | 99 | 2 | 97 | 101 |
| 30 | 99 | 2 | 97 | 101 |
| 40 | 99 | 2 | 97 | 101 |
| 50 | 99 | 2 | 97 | 101 |
| 60 | 99 | 2 | 97 | 101 |

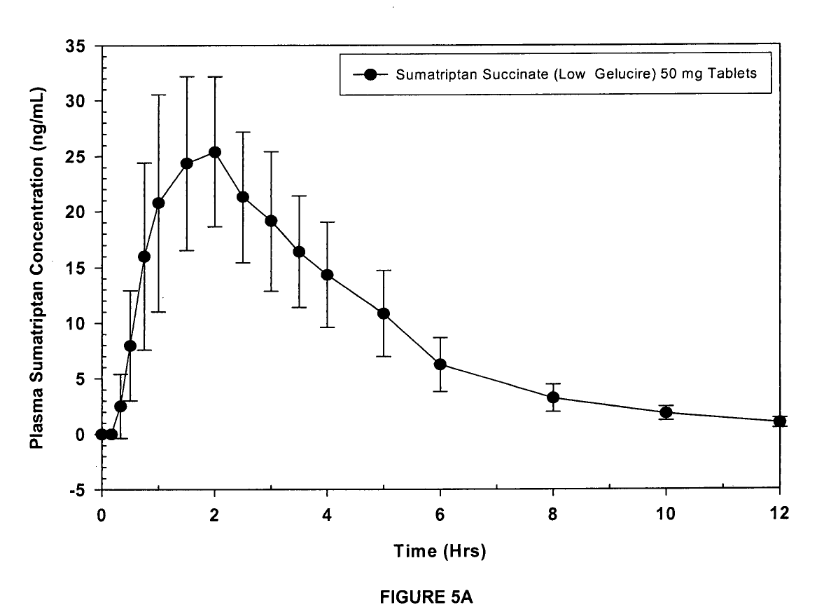

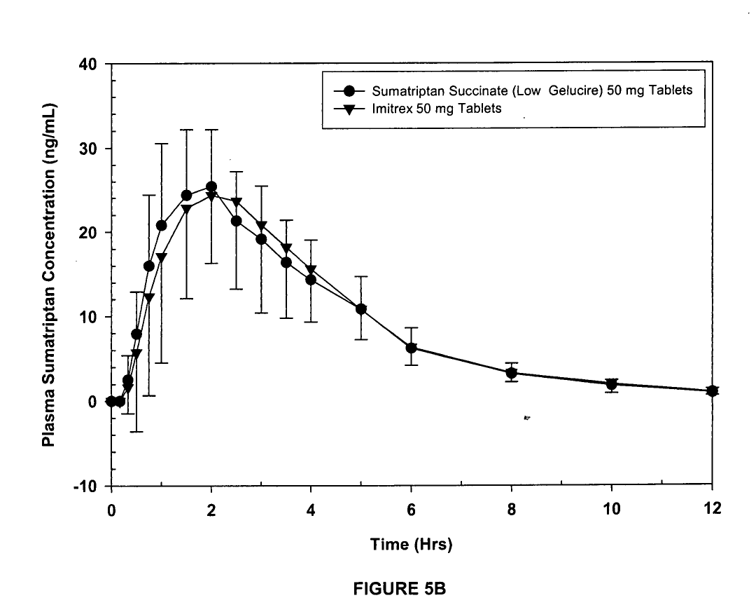

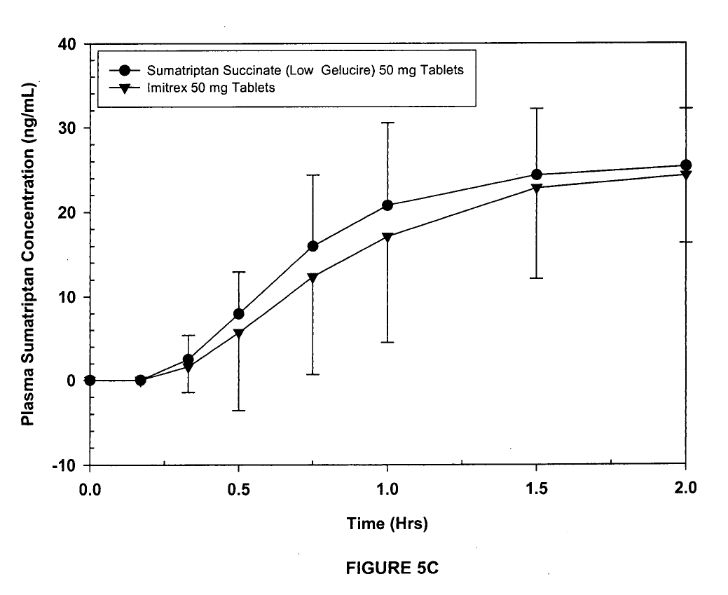

The dissolution profile of the Imitrex® tablet is shown in FIG. 5

### EXAMPLE 11

Conventional Directly Compressible Tablet:

The uncoated microparticles as prepared in Example 1 were used in the following tablet composition:

| | % w/w of 100 mg |
|---|---|
| Tablet Component | Tablet |
| Sumatriptan Succinate coated | 52.00 |
| microparticles (low macrogol | |
| fatty acid ester content) | |
| Lactose Supertab | 9.50 |
| Monohydrate | |
| Microcrystalline Cellulose^{a} | 35.5 |
| Kollidon CL | 2.00 |
| Silicon dioxide^{b} | 0.50 |
| Magnesium Stearate | 0.50 |
| Total | 100.00 |

^{a}Avicel ® PH101
^{b}Syloid ® 244FP

All of the microcrystalline cellulose, microparticles, lactose and Kollidon CL is placed in a Turbula mixer and mixed for about 2 minutes. All of the silicon dioxide is next added and the entire blend is mixed for about 1 minute. All of the magnesium stearate is next added and mixed for another 1 minute.

The blend is next compressed to a target weight of 903 mg in an F-press using a 15 mm diameter tooling. The resulting tablet typically has a hardness value of about 96 N, a thickness of about 5.12 mm and a friability of about 0.1%.

The dissolution profile of the tablet is determined as described in Examples 5-7 and produced the following dissolution profile:

| Time | Mean | Std. Dev. | Min. | Max. |
|---|---|---|---|---|
| (minutes) | (%) | (%) | (%) | (%) |
| 0 | 0 | 0 | 0 | 0 |
| 10 | 93 | 1 | 92 | 96 |
| 20 | 102 | 1 | 100 | 103 |
| 30 | 102 | 1 | 101 | 104 |
| 40 | 102 | 1 | 101 | 104 |
| 50 | 103 | 1 | 102 | 105 |
| 60 | 104 | 1 | 103 | 106 |

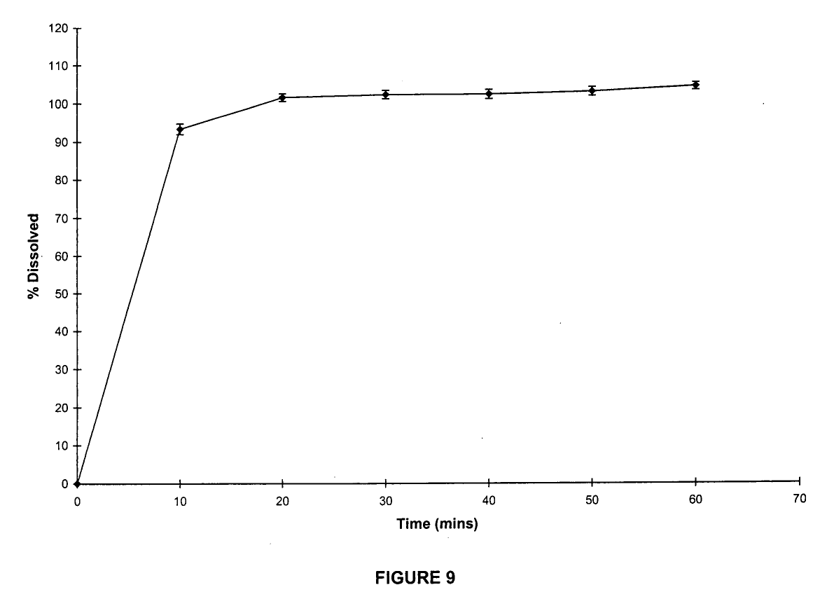

The dissolution profile of the conventional direct compression tablet is show in FIG. 9.

### EXAMPLE 12

Comparative Study of the Bioavailability of Sumatriptan:

A comparative study was conducted to determine the bioavailability of sumatriptan following a single-dose tablet between the tablets generated in Examples 5, 6 and the prior art Imitrex® tablet (50 mg).

For all three studies, the 18 subjects were requested to complete a light breakfast consisting of one bran muffin and 180 ml of homogenized milk, one hour to administration of the tablet.

Subject received one of the following treatments at 0.0 hours on Day 1 of each of the three study periods, according to a randomized scheme:

Treatment A (for 50 mg Tablets Described in Examples 5 and 6):

One hour following the completion of the light breakfast, one tablet from either sample 5 or 6 was placed directly on the tongue and the subjects were requested to suck on the tablet for about 1 minute until completely dissolved. Subjects were instructed not to swallow or chew any portion of the tablet. The subject's mouth was then checked to ensure that the tablet has completely dissolved. If the tablet has not completely dissolved, the subject was instructed to suck on the tablet until the tablet has completely dissolved. A check of each subject's mouth was made again to ensure drug ingestion. The subjects were then requested to consume 60 ml of ambient temperature water. The subjects were then requested to consume one regular sized oatmeal cookie followed by 120 ml of ambient temperature water. All procedures were completed within seven minutes. The actual dosing time was recorded when the tablet was placed on the subject's tongue.

Treatment B (for 50 mg Prior Art Imitrex® Tablet):

One hour following the completion of a light breakfast, one Imitrex® 50 mg tablet was administered with 60 ml of ambient temperature water. A check of each subject's mouth was made to ensure tablet ingestion. The subjects were then requested to consume one regular sized oatmeal cookie followed by 120 ml of ambient temperature water, both of which must be consumed within five minutes. The Imitrex® tablet was to be swallowed whole, not chewed.

Table 1 below summarizes the mean plasma sumatriptan concentrations (ng/ml) over a 12-hour period after administration of the respective dosage forms:

TABLE 1

| | Sumatriptan | Sumatriptan | |
|---|---|---|---|
| | Succinate | Succinate | |
| | (Low macrogol | (High macrogol | |
| | fatty acid ester | fatty acid ester | Imitrex ® |
| Time | content) 50 mg | content) 50 mg | 50 mg |
| (Hrs) | Tablets | Tablets | Tablets |
| 0 | 0.00 ± 0.00 | 0.00 ± 0.00 | 0.00 ± 0.00 |
| 0.17 | 0.00 ± 0.00 | 0.20 ± 0.40 | 0.00 ± 0.00 |
| 0.33 | 2.51 ± 2.89 | 4.27 ± 3.55 | 1.60 ± 3.05 |
| 0.5 | 7.96 ± 4.96 | 10.35 ± 5.76 | 5.71 ± 9.31 |
| 0.75 | 15.99 ± 8.42 | 18.99 ± 8.44 | 12.33 ± 11.65 |
| 1.0 | 20.78 ± 9.76 | 21.13 ± 8.76 | 17.13 ± 12.61 |
| 1.5 | 24.35 ± 7.82 | 25.86 ± 7.59 | 22.81 ± 10.70 |
| 2.0 | 25.39 ± 6.76 | 24.87 ± 7.37 | 24.32 ± 8.00 |
| 2.5 | 21.31 ± 5.89 | 22.35 ± 7.05 | 23.62 + 10.42 |
| 3.0 | 19.14 ± 6.29 | 19.54 ± 6.19 | 20.84 ± 10.45 |
| 3.5 | 16.39 ± 5.00 | 16.15 ± 4.94 | 18.18 ± 8.40 |
| 4.0 | 14.32 ± 4.71 | 13.89 ± 4.05 | 15.60 ± 6.26 |
| 5.0 | 10.83 ± 3.88 | 10.16 + 3.59 | 10.77 ± 3.54 |
| 6.0 | 6.21 ± 2.42 | 6.10 ± 2.15 | 6.34 ± 2.13 |
| 8.0 | 3.20 ± 1.22 | 3.04 ± 0.97 | 3.28 ± 1.03 |
| 10.0 | 1.82 ± 0.62 | 1.76 ± 0.65 | 2.01 ± 1.11 |
| 12.0 | 0.96 ± 0.44 | 1.01 ± 0.48 | 1.00 + 0.38 |

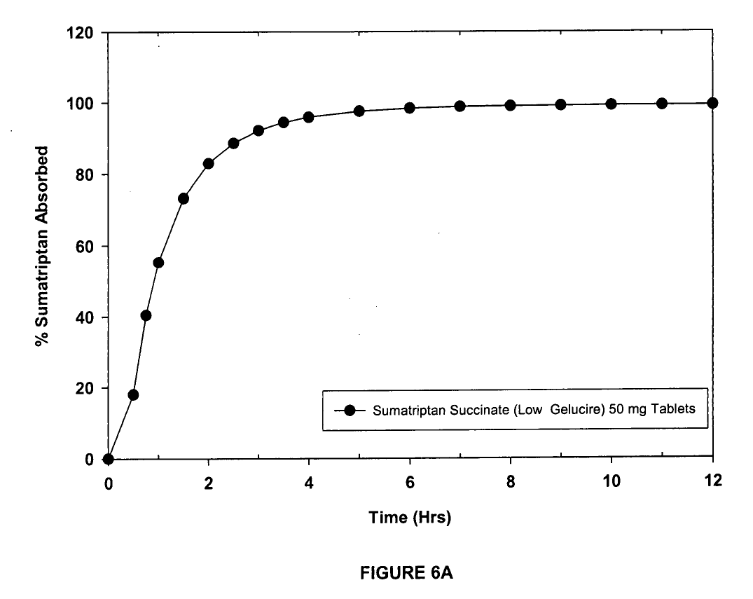

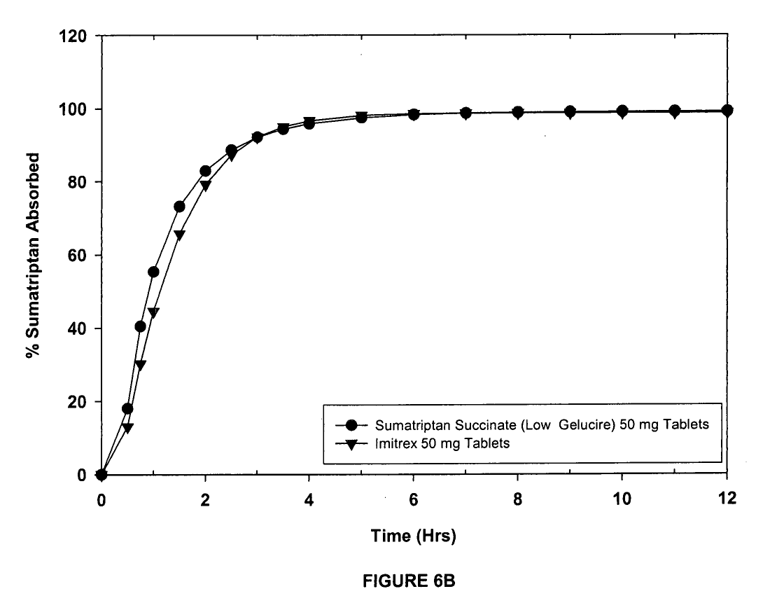

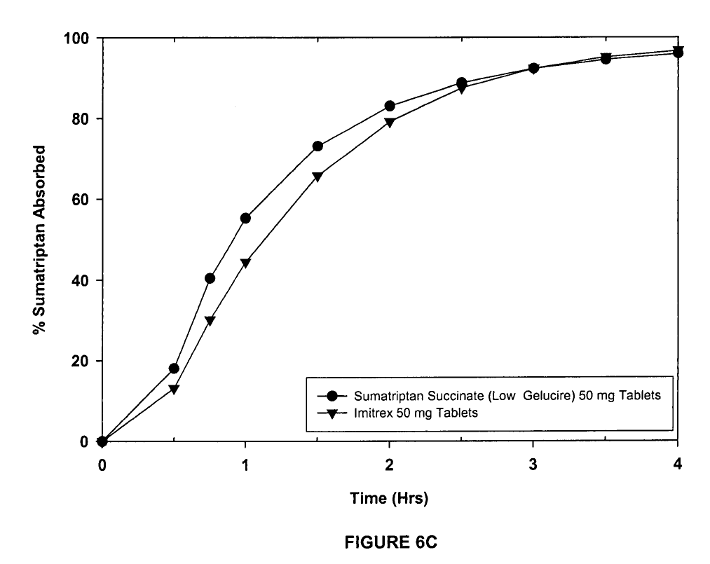

The corresponding sumatriptan plasma-concentration profiles of the tablets with low macrogol fatty acid ester content or high macrogol fatty acid ester content are shown either alone in FIGS. 4A and 6A respectively or in comparison with the plasma-concentration profile of the prior art Imitrex® tablet in FIGS. 4B-4C and FIGS. 6B-6C respectively.

A comparison of the mean in-vivo absorption rate of the sumatriptan tablets according to Example 5 and 6 with that of the prior art 50 mg Imitrex® tablet can be determined from the data in Table 1 using the Wagner-Nelson numerical deconvolution method, a statistical method well known in the art and recognized by the US Food and Drug Administration. Table 3 summarizes the comparison of the absorption data:

TABLE 3

| | Sumatriptan Succinate | | Sumatriptan Succinate | | | |
|---|---|---|---|---|---|---|
| | (Low Gelucire) | | (High Gelucire) | | Imitrex ® | |
| | 50 mg Tablets | | 50 mg Tablets | | 50 mg Tablets | |
| | Concentration | % | Concentration | % | Concentration | % |
| Time (Hrs) | (ng/ml) | Absorbed | (ng/ml) | Absorbed | (ng/ml) | Absorbed |
| 0 | 0.00 | 0.0 | 0.00 | 0.0 | 0.00 | 0.0 |
| 0.5 | 7.96 | 18.1 | 10.35 | 25.4 | 5.71 | 13.0 |
| 0.75 | 15.99 | 40.4 | 18.99 | 44.9 | 12.33 | 30.1 |
| 1.0 | 20.78 | 55.3 | 21.13 | 58.7 | 17.13 | 44.5 |
| 1.5 | 24.35 | 73.2 | 25.86 | 76.0 | 22.81 | 65.7 |
| 2.0 | 25.39 | 82.9 | 24.87 | 85.5 | 24.32 | 79.2 |
| 2.5 | 21.31 | 88.7 | 22.35 | 90.9 | 23.62 | 87.4 |
| 3.0 | 19.14 | 92.2 | 19.54 | 94.0 | 20.84 | 92.2 |
| 3.5 | 16.39 | 94.4 | 16.15 | 95.9 | 18.18 | 95.1 |
| 4.0 | 14.32 | 95.9 | 13.89 | 96.9 | 15.60 | 96.7 |
| 5.0 | 10.83 | 97.5 | 10.16 | 98.0 | 10.77 | 98.1 |
| 6.0 | 6.21 | 98.3 | 6.10 | 98.4 | 6.34 | 98.5 |
| 8.0 | 3.20 | 98.9 | 3.04 | 98.6 | 3.28 | 98.7 |
| 10.0 | 1.82 | 99.0 | 1.76 | 98.6 | 2.01 | 98.7 |
| 12.0 | 0.96 | 99.1 | 1.01 | 98.6 | 1.00 | 98.7 |

| | | | |
|---|---|---|---|
| Time Taken | 0.90 | 0.83 | 1.11 |
| for 50% of | | | |
| Sumatriptan to | | | |
| be absorbed | | | |
| (T₅₀) (hrs) | | | |

Tables 4 and 5 provide the mean pharmacokinetic parameters for sumatriptan following administration of the tablets of Examples 5 and 6 respectively in comparison with that of the prior art Imitrex® 50 mg tablet:

TABLE 4

| | Sumatriptan Succinate (Low | | | | | | | | | |
|---|---|---|---|---|---|---|---|---|---|---|
| | macrogol fatty acid ester | | | | | | | | | |
| | content) 50 mg Tablets | | | | | Imitrex ® 50 mg Tablets | | | | |
| Subject | AUC₍₀₋ₜ₎ | AUC_{(0-inf)} | Cₘₐₓ | Tₘₐₓ | T_{half} | AUC₍₀₋ₜ₎ | AUC_{(0-inf)} | Cₘₐₓ | Tₘₐₓ | T_{half} |
| 1 | 90.61 | 92.18 | 22.20 | 1.50 | 1.86 | 90.28 | 91.97 | 24.44 | 2.00 | 1.93 |
| 2 | 81.80 | 83.83 | 15.12 | 2.00 | 2.00 | 73.89 | 76.06 | 16.39 | 3.00 | 1.93 |
| 3 | 126.16 | 132.12 | 25.36 | 1.50 | 2.53 | 101.94 | 105.71 | 18.37 | 2.00 | 2.10 |
| 4 | 71.91 | 74.37 | 16.69 | 1.00 | 2.55 | 68.50 | 70.76 | 15.04 | 1.50 | 2.46 |
| 5 | 144.70 | 148.55 | 37.71 | 1.50 | 2.14 | 182.90 | 186.67 | 55.45 | 1.50 | 2.35 |
| 7 | 71.78 | 73.92 | 16.72 | 1.00 | 2.26 | 79.24 | 81.21 | 20.42 | 2.00 | 1.99 |
| 8 | 152.47 | 155.51 | 35.65 | 3.00 | 1.82 | 175.90 | 179.14 | 54.04 | 2.50 | 2.11 |
| 10 | 127.01 | 131.16 | 25.84 | 2.00 | 2.16 | 107.90 | 111.22 | 22.88 | 2.00 | 2.18 |
| 11 | 108.19 | 112.47 | 25.52 | 1.50 | 2.32 | 106.11 | 112.27 | 35.24 | 1.00 | 2.93 |
| 12 | 104.24 | 109.08 | 29.48 | 2.00 | 2.72 | 110.52 | 120.57 | 24.71 | 2.00 | 3.65 |
| 13 | 112.13 | 113.93 | 35.66 | 1.00 | 1.93 | 94.17 | 96.49 | 26.73 | 2.00 | 2.20 |
| 14 | 121.36 | 127.42 | 28.71 | 2.00 | 2.52 | 133.89 | 138.71 | 28.23 | 2.00 | 2.27 |
| 15 | 100.36 | 101.90 | 28.86 | 2.00 | 1.88 | 93.63 | 95.75 | 24.13 | 2.50 | 2.15 |
| 16 | 163.38 | 166.25 | 46.20 | 1.00 | 1.96 | 149.34 | 152.30 | 36.71 | 3.00 | 1.96 |
| 17 | 68.72 | 70.29 | 26.32 | 2.00 | 1.92 | 91.23 | 92.99 | 35.97 | 1.50 | 2.31 |
| 18 | 100.08 | 103.20 | 25.60 | 2.00 | 2.16 | 90.85 | 94.06 | 21.10 | 2.00 | 2.40 |
| Mean | 109.06 | 112.26 | 27.60 | 1.69 | 2.17 | 109.39 | 112.87 | 28.74 | 2.03 | 2.31 |
| SD | 28.92 | 29.63 | 8.26 | 0.54 | 0.29 | 34.11 | 34.69 | 12.06 | 0.53 | 0.44 |
| CV | 26.51 | 26.39 | 29.94 | 32.23 | 13.16 | 31.18 | 30.74 | 41.96 | 26.16 | 18.97 |
| Min | 68.72 | 70.29 | 15.12 | 1.00 | 1.82 | 68.50 | 70.76 | 15.04 | 1.00 | 1.93 |
| Max | 163.38 | 166.25 | 46.20 | 3.00 | 2.72 | 182.90 | 186.67 | 55.45 | 3.00 | 3.65 |

TABLE 5

| | Sumatriptan Succinate (High | | | | | | | | | |
|---|---|---|---|---|---|---|---|---|---|---|
| | macrogol fatty acid ester | | | | | | | | | |
| | content) 50 mg Tablets | | | | | Imitrex ® 50 mg Tablets | | | | |
| Subject | AUC₍₀₋ₜ₎ | AUC_{(0-inf)} | Cₘₐₓ | Tₘₐₓ | T_{half} | AUC₍₀₋ₜ₎ | AUC_{(0-inf)} | Cₘₐₓ | Tₘₐₓ | T_{half} |
| 1 | 108.95 | 110.79 | 28.64 | 1.50 | 1.95 | 90.28 | 91.97 | 24.44 | 2.00 | 1.93 |
| 2 | 91.08 | 93.55 | 19.80 | 1.50 | 2.06 | 73.89 | 76.06 | 16.39 | 3.00 | 1.93 |
| 3 | 113.71 | 119.51 | 23.54 | 1.50 | 2.61 | 101.94 | 105.71 | 18.37 | 2.00 | 2.10 |
| 4 | 59.95 | 62.16 | 14.34 | 1.00 | 2.43 | 68.50 | 70.76 | 15.04 | 1.50 | 2.46 |
| 5 | 165.18 | 169.54 | 46.19 | 1.00 | 2.21 | 182.90 | 186.67 | 55.45 | 1.50 | 2.35 |
| 7 | 75.93 | 78.16 | 16.52 | 1.50 | 2.02 | 79.24 | 81.21 | 20.42 | 2.00 | 1.99 |
| 8 | 163.06 | 165.79 | 37.30 | 1.50 | 1.84 | 175.90 | 179.14 | 54.04 | 2.50 | 2.11 |
| 10 | 130.94 | 134.62 | 25.38 | 1.50 | 2.13 | 107.90 | 111.22 | 22.88 | 2.00 | 2.18 |
| 11 | 94.19 | 100.11 | 21.52 | 1.50 | 2.83 | 106.11 | 112.27 | 35.24 | 1.00 | 2.93 |
| 12 | 122.99 | 131.05 | 28.78 | 2.00 | 2.97 | 110.52 | 120.57 | 24.71 | 2.00 | 3.65 |
| 13 | 99.48 | 101.74 | 28.53 | 0.75 | 2.34 | 94.17 | 96.49 | 26.73 | 2.00 | 2.20 |
| 14 | 125.07 | 131.10 | 25.83 | 2.00 | 2.62 | 133.89 | 138.71 | 28.23 | 2.00 | 2.27 |
| 15 | 118.51 | 120.47 | 30.59 | 2.00 | 2.06 | 93.63 | 95.75 | 24.13 | 2.50 | 2.15 |
| 16 | 127.07 | 129.92 | 32.91 | 2.00 | 2.43 | 149.34 | 152.30 | 36.71 | 3.00 | 1.96 |
| 17 | 70.36 | 72.03 | 20.50 | 2.00 | 2.16 | 91.23 | 92.99 | 35.97 | 1.50 | 2.31 |
| 18 | 98.13 | 102.05 | 26.64 | 1.50 | 2.60 | 90.85 | 94.06 | 21.10 | 2.00 | 2.40 |
| Mean | 110.29 | 113.91 | 26.69 | 1.55 | 2.33 | 109.39 | 112.87 | 28.74 | 2.03 | 2.31 |
| SD | 29.63 | 30.28 | 7.91 | 0.39 | 0.33 | 34.11 | 34.69 | 12.06 | 0.53 | 0.44 |
| CV | 26.86 | 26.58 | 29.65 | 25.19 | 14.12 | 31.18 | 30.74 | 41.96 | 26.16 | 18.97 |
| Min | 59.95 | 62.16 | 14.34 | 0.75 | 1.84 | 68.50 | 70.76 | 15.04 | 1.00 | 1.93 |
| Max | 165.18 | 169.54 | 46.19 | 2.00 | 2.97 | 182.90 | 186.67 | 55.45 | 3.00 | 3.65 |

TABLE 6

| | Sumatriptan Succinate | | Sumatriptan Succinate | |
|---|---|---|---|---|
| | (Low macrogol fatty acid ester | | (High macrogol fatty acid ester | |
| | content) vs. Imitrex ® | | content) vs. Imitrex ® | |
| | Ratio | 90% Cl | Ratio | 90% Cl |
| AUC | 1.00 | 0.94-1.07 | 0.99 | 0.95-1.08 |
| Cₘₐₓ | 0.99 | 0.89-1.09 | 0.96 | 0.86-1.06 |

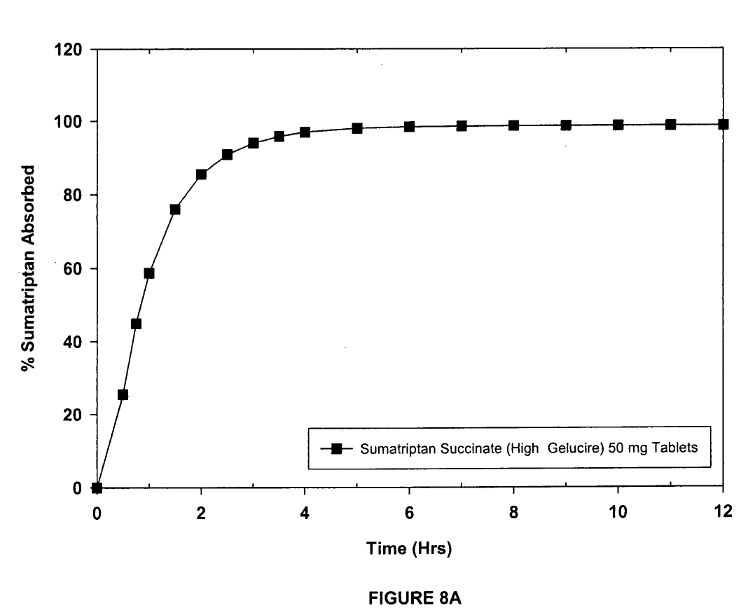

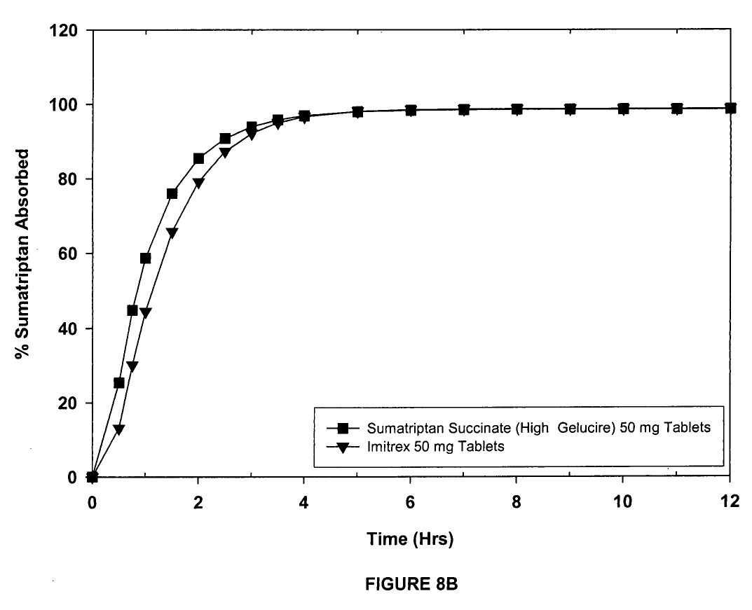

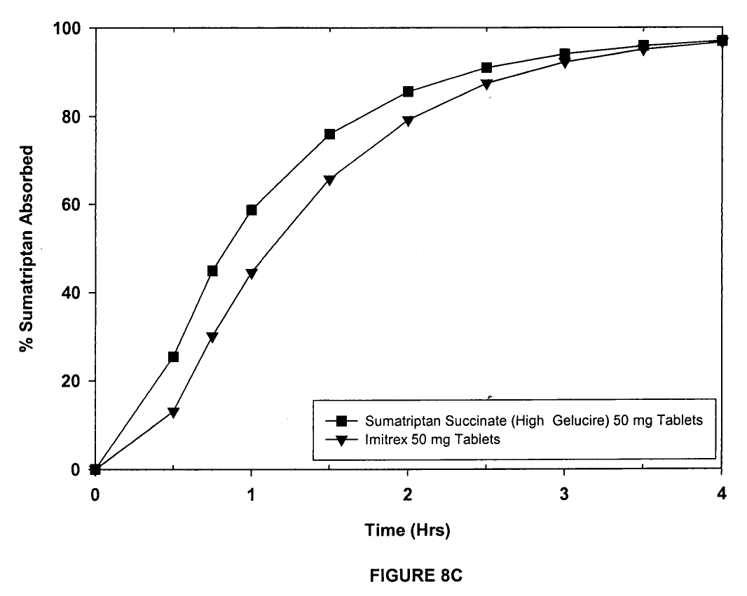

The results reported in Tables 1-6, and shown in FIGS. 5A-8C demonstrate that there is a significant enhancement in the in-vivo rate of absorption of sumatriptan comprised in the composition of the instant invention regardless of the percentage of macrogol fatty acid ester present when compared to the rate of absorption of sumatriptan in the prior art Imitrex® tablet while remaining bioequivalent to Imitrex®. This is in spite of the differences in the in-vitro dissolution data of the compositions of the instant invention when compared to the prior art Imitrex® tablet. Thus, while the tablet comprising the low macrogol fatty acid content coated microparticles showed a slower dissolution profile in comparison to the tablet comprising the high macrogol fatty acid ester content coated microparticles with respect to Imitrex®, both showed a faster in-vivo absorption rate for sumatriptan with respect to Imitrex®. These results are particularly surprising and demonstrate that in this particular instance, there is no correlation between the in-vitro dissolution data and in-vivo absorption rate of sumatriptan.

The absorption data presented herein is also surprising in view data presented by Fuseau et al. (Clinical Therapeutics, pp 242-251:23, 2001). This paper evaluated in one study the absorption and bioequivalence of a conventional 50 mg sumatriptan tablet and an encapsulated 50 mg sumatriptan tablet in healthy individuals not suffering a migraine. The data presented therein clearly show that encapsulated sumatriptan tablets delay the absorption of sumatriptan. Absorption of sumatriptan was reduced by 21% with the encapsulated sumatriptan tablet over the interval from dosing to 2 hours. The lower bounds of the 90% CIs for the encapsulated tablet/conventional tablet ratios lay outside the traditional bounds for bioequivalence (0.8-1.25). The encapsulated tablet/conventional tablet ratio of the geometric mean in healthy volunteers is 0.79 (90% CI, 0.588-1.050). In contrast, the composition of the invention described herein exhibits a faster rate of absorption over the interval from dosing to 2 hours but remain bioequivalent to Imitrex® as demonstrated by the 90% CIs for the ratios of the tablets comprising compositions of the invention to Imitrex®, which lie within the traditional bounds for bioequivalence (0.8-1.25).

In summary, the data presented herein demonstrate that pharmaceutical compositions of the instant invention comprising at least 5% macrogol fatty acid ester significantly enhances the in-vivo absorption rate of sumatriptan while remaining bioequivalent to Imitrex®.

While certain preferred and alternative embodiments of the invention have been set forth for purposes of disclosing the invention, modifications to the disclosed embodiments may occur to those who are skilled in the art. Accordingly, the appended claims are intended to cover all embodiments of the invention and modifications thereof, which do not depart from the spirit and scope of the invention.

1. A rapid absorption pharmaceutical composition comprising an effective amount of at least one selective 5-HT agonist, at least one spheronization aid and at least one solubility enhancer.

2. The rapid absorption pharmaceutical composition of claim 1 wherein said composition is incorporated into a plurality of microparticles.

3. The rapid absorption pharmaceutical composition of claim 2 wherein each microparticle is from about 150 μm to about 500 μm in diameter.

4. The rapid absorption pharmaceutical composition of claim 3 wherein each microparticle is from about 200 μm to about 250 μm in diameter.

5. The rapid absorption pharmaceutical composition of claim 4 wherein said at least one selective 5-HT agonist is selected form the group consisting of sumatriptan, zolmitriptan, rizatriptan, naratriptan, frovatriptan, eletriptan, almotriptan and any combination thereof.

6. The rapid absorption pharmaceutical composition of claim 5 wherein said at least one selective 5-HT agonist is sumatriptan.

7. The rapid absorption pharmaceutical composition of claim 6 wherein said sumatriptan is present in an amount from about 1% to about 60% by weight of each said microparticle.

8. The rapid absorption pharmaceutical composition of claim 7 wherein said sumatriptan is present in an amount from about 20% to about 50% by weight of each said microparticle.

9. The rapid absorption pharmaceutical composition of claim 8 wherein said sumatriptan is present in an amount from about 30% to about 40% by weight of each said microparticle.

10. The rapid absorption pharmaceutical composition of claim 4 wherein said at least one spheronization aid is selected from the group consisting of distilled monoglycerides, glyceryl behenate, glyceryl palmitostearate, hydrogenated vegetable oils, polyoxyethylene ethers, cetostearyl alcohol, thermo-softening polymers and any combination thereof.

11. The rapid absorption pharmaceutical composition of claim 10 wherein said at least one spheronization aid is glyceryl palmitostearate.

12. The rapid absorption pharmaceutical composition of claim 11 wherein said glyceryl palmitostearate is present in an amount from about 5% to about 90% by weight of each microparticle.

13. The rapid absorption pharmaceutical composition of claim 12 wherein said glyceryl palmitostearate is present in an amount from about 15% to about 75% by weight of each microparticle.

14. The rapid absorption pharmaceutical composition of claim 13 wherein said distilled glyceryl palmitostearate is present in an amount from about 25% to about 45% by weight of each microparticle.

15. The rapid absorption pharmaceutical composition of claim 14 wherein said distilled glyceryl palmitostearate is present in an amount of about 35% by weight of each microparticle.

16. The rapid absorption pharmaceutical composition of claim 4 wherein said at least one solubility enhancer is selected from the group consisting of a macrogol fatty acid ester, poloxamer, polyethylene glycol, polyvinylpyrrolidone, sodium lauryl sulfate, and any combination thereof.

17. The rapid absorption pharmaceutical composition of claim 16 wherein said at least one solubility enhancer is a macrogol fatty acid ester.

18. The rapid absorption pharmaceutical composition of claim 17 wherein said macrogol fatty acid ester is in an amount greater than from about 0% to about 95% by weight of each microparticle.

19. The rapid absorption pharmaceutical composition of claim 18 wherein said macrogol fatty acid ester is present in an amount from about 1% to about 50% by weight of each microparticle.

20. The rapid absorption pharmaceutical composition of claim 19 wherein said macrogol fatty acid ester is present in an amount of from about 5% to about 35% by weight of each microparticle.

21. The rapid absorption pharmaceutical composition of claim 20 wherein said macrogol fatty acid ester is present in an amount of about 5% by weight of each microparticle.

22. The rapid absorption pharmaceutical composition of claim 20 wherein said macrogol fatty acid ester is present in an amount of about 35% by weight of each microparticle.

23. The rapid absorption pharmaceutical composition of claim 17 wherein said macrogol fatty acid ester is selected from the group consisting of Gelucire 50/13, Gelucire 44/14 and any combination thereof.

24. The rapid absorption pharmaceutical composition of claim 23 wherein said macrogol fatty acid ester is Gelucire 50/13.

25. The rapid absorption pharmaceutical composition of claim 21 wherein said macrogol fatty acid ester is Gelucire 50/13.

26. The rapid absorption pharmaceutical composition of claim 22 wherein said macrogol fatty acid ester is Gelucire 50/13.

27. The rapid absorption pharmaceutical composition of claim 4 wherein said microparticles are coated with at least one taste-masking coat.

28. The rapid absorption pharmaceutical composition of claim 27 wherein the at least one taste-masking coating is comprised of a combination of at least one hydrophobic polymer and at least one hydrophilic polymer.

29. The rapid absorption pharmaceutical composition of claim 28 wherein the hydrophobic polymer and hydrophilic polymer is present in a ratio of 7:3 respectively.

30. The rapid absorption pharmaceutical composition of claim 29 wherein said hydrophobic polymer is Ethylcellulose E45 and said hydrophilic polymer is Povidone K30.

31. A rapid absorption pharmaceutical composition comprising an effective amount of a selective 5-HT agonist sumatriptan, glyceryl palmitostearate, and a macrogol fatty acid ester.

32. The rapid absorption pharmaceutical composition of claim 31 wherein said composition is in the form of a plurality of microparticles.

33. The rapid absorption pharmaceutical composition of claim 32 wherein said microparticles are coated with a taste-masking coating.

34. The rapid absorption pharmaceutical composition of claim 33 wherein said sumatriptan is about 30% by weight of each microparticle, said glyceryl palmitostearate is about 65% by weight of each microparticle and said macrogol fatty acid ester is about 5% by weight of each microparticle.

35. The rapid absorption pharmaceutical composition of claim 34 wherein said macrogol fatty acid ester is Gelucire 50/13.

36. The rapid absorption pharmaceutical composition of claim 33 wherein said sumatriptan is about 30% by weight of each microparticle, said glyceryl palmitostearate is about 35% by weight of each microparticle and said macrogol fatty acid ester is about 35% by weight of each microparticle.

37. The rapid absorption pharmaceutical composition of claim 36 wherein said macrogol fatty acid ester is Gelucire 50/13.

38. The rapid absorption pharmaceutical composition of claim 35 wherein said microparticles are incorporated into a suitable oral dosage form.

39. The rapid absorption pharmaceutical composition of claim 38 wherein said oral dosage form is selected from the group consisting of a fast-dispersing direct compression non-cushioning matrix tablet, a fast-dispersing direct compression cushioning matrix tablet, a direct compression non-cushioning matrix tablet, a direct compression cushioning matrix tablet, capsule, buccal tablet, and sachet.

40. The rapid absorption pharmaceutical composition of claim 39 wherein said oral dosage form is a fast-dispersing direct compression non-cushioning matrix tablet.

41. The rapid absorption pharmaceutical composition of claim 37 wherein said microparticles are incorporated into a suitable oral dosage form.

42. The rapid absorption pharmaceutical composition of claim 41 wherein said oral dosage form is selected from the group consisting of a fast-dispersing direct compression non-cushioning matrix tablet, a fast-dispersing direct compression cushioning matrix tablet, a direct compression non-cushioning matrix tablet, a direct compression cushioning matrix tablet, capsule, buccal tablet, and sachet.

43. The rapid absorption pharmaceutical composition of claim 42 wherein said oral dosage form is a fast-dispersing direct compression non-cushioning matrix tablet.

44. The use of the rapid absorption pharmaceutical composition of claim 1 for the manufacture of a medicament for the treatment of migraine.

45. The use of the rapid absorption pharmaceutical composition of claim 4 for the manufacture of a medicament for the treatment of migraine.

46. The use of the rapid absorption pharmaceutical composition of claim 35 for the manufacture of a medicament for the treatment of migraine.

47. The use of the rapid absorption pharmaceutical composition of claim 37 for the manufacture of a medicament for the treatment of migraine.

48 An oral dosage form comprising:
(a) a plurality of microparticles coated with at least one taste-masking coating, said microparticles comprising a rapid absorption composition of an effective amount of at least one selective 5-HT agonist, at least one spheronization aid, and at least one solubility enhancer, and
(b) a non-cushioning matrix,
wherein said taste-masked coated microparticles are dispersed within said matrix and said dosage form is adapted to rapidly dissolve in the mouth of a patient.

49. The oral dosage form of claim 48 wherein said non-cushioning matrix comprises at least one linear polyol and/or a lactose or maltose and optionally an inorganic salt, cellulose or a disintegrant or any mixture of an inorganic salt, cellulose or disintegrant.

50. The oral dosage form of claim 49 wherein said linear polyol, and said optional inorganic salt or cellulose are of directly compressible grades.

51. The oral dosage form of claim 50 wherein said linear polyol is selected from the group consisting of powdered mannitol, powdered sorbitol, powdered xylitol, directly compressible mannitol, directly compressible sorbitol, directly compressible xylitol and any combination thereof.

52 The oral dosage form of claim 51 wherein said linear polyol is directly compressible mannitol.

53. The oral dosage form of claim 52 wherein said polyol is present in an amount from about greater than 0% to about 85% by weight of the dosage form.

54. The oral dosage form of claim 53 wherein said polyol is present in an amount from about 20% to about 60% by weight of the dosage form.

55. The oral dosage form of claim 54 wherein said polyol is present in an amount from about 40% to about 50% by weight of the dosage form.

56. The oral dosage form of claim 49 wherein said optional inorganic salt is selected from the group consisting of powdered calcium carbonate, powdered dibasic anhydrous calcium phosphate, powdered dibasic dihydrate calcium phosphate, powdered tribasic calcium phosphate, powdered dihydrate calcium sulfate, powdered monobasic sodium phosphate, powdered dibasic sodium phosphate, powdered anhydrous magnesium carbonate, powdered alkaline magnesium oxide, directly compressible grades of calcium carbonate, directly compressible grades of dibasic anhydrous calcium phosphate, directly compressible grades of dibasic calcium phosphate dihydrate, directly compressible grades of tribasic calcium phosphate, directly compressible grades of calcium sulfate, directly compressible grades of anhydrous magnesium carbonate, directly compressible grades of magnesium aluminum silicate NF, directly compressible grades of alkaline magnesium oxide and any combination thereof.

57. The oral dosage form of claim 56 wherein said optional inorganic salt is directly compressible grades of dibasic calcium phosphate dihydrate.

58. The oral dosage form of claim 57 wherein said optional inorganic salt is present in an amount from about 0% to about 50% by weight of the dosage form.

59. The oral dosage form of claim 58 wherein said optional inorganic salt is present in an amount from about 5% to about 30% by weight of the dosage form.

60. The oral dosage form of claim 59 wherein said optional inorganic salt is present in an amount from about 7% to about 15% by weight of the dosage form.

61. The oral dosage form of claim 49 wherein said optional cellulose is selected from the group consisting of powdered cellulose, powdered silicified microcrystalline, powdered microcrystalline cellulose, directly compressible grades of silicified microcrystalline cellulose, directly compressible grades of microcrystalline cellulose and any combination thereof.

62. The oral dosage form of claim 61 wherein said optional cellulose is directly compressible grades of microcrystalline cellulose.

63. The oral dosage form of claim 62 wherein said optional cellulose is present in an amount from about 0% to about 40% by weight of the dosage form.

64. The oral dosage form of claim 63 wherein said optional cellulose is present in an amount from about 5% to about 30% by weight of the dosage form.

65. The oral dosage form of claim 64 wherein said optional cellulose is present in an amount from about 10% to about 20% by weight of the dosage form.

66. The oral dosage form of claim 49 wherein said optional disintegrant is selected from the group consisting of crospovidone, croscarmellose sodium, sodium starch glycolate, sodium starch glycolate (crosslinked low substituted), sodium starch glycolate (highly crosslinked), hydroxypropyl cellulose (low substituted), polacrilin potassium, pregelatinized starch, microcrystalline cellulose, and any combination thereof.

67. The oral dosage form of claim 66 wherein said optional disintegrant is crospovidone.

68. The oral dosage form of claim 67 wherein said optional disintegrant is present in an amount from about 0% to about 3% by weight of the dosage form.

69. The oral dosage form of claim 68 wherein said optional disintegrant is present in an amount from about 2% to about 3% by weight of the dosage form.

70. The oral dosage form of claim 69 wherein said optional disintegrant is present in an amount from about 2.5% to about 3% by weight of the dosage form.

71. The oral dosage form of claim 49 wherein said at least one selective 5-HT agonist is selected from the group consisting of sumatriptan, zolmitriptan, rizatriptan, naratriptan, frovatriptan, eletriptan, almotriptan and any combination thereof.

72. The oral dosage form of claim 71 wherein said at least one selective 5-HT agonist is sumatriptan.

73. The oral dosage form of claim 72 wherein said sumatriptan is present in an amount of from about 1% to about 60% by weight of each microparticle.

74. The oral dosage form of claim 73 wherein said sumatriptan is present in an amount from about 20% to about 50% by weight of each microparticle.

75. The oral dosage form of claim 74 wherein said sumatriptan is present in an amount of from about 30% to about 40% by weight of each microparticle.

76. The oral dosage form of claim 49 wherein said at least one spheronization aid is selected from the group consisting of distilled monoglycerides, glyceryl behenate, glyceryl palmitostearate, hydrogenated vegetable oils, polyoxyethylene ethers, cetostearyl alcohol, thermo-softening polymers and any combination thereof.

77. The oral dosage form of claim 76 wherein said at least one spheronization aid is glyceryl palmitostearate.

78. The oral dosage form of claim 77 wherein said glyceryl palmitostearate is present in an amount from about 5% to about 90% by weight of each microparticle.

79. The oral dosage form of claim 78 wherein said glyceryl palmitostearate is present in an amount from about 15% to about 75% by weight of each microparticle.

80. The oral dosage from of claim 79 wherein said glyceryl palmitostearate is present in an amount from about 25% to about 45% by weight of each microparticle.

81. The oral dosage form of claim 49 wherein said at least one solubility enhancer is selected form the group consisting of a macrogol fatty acid ester, poloxamer, polyethylene glycol, polyvinylpyrrolidones, sodium lauryl sulfate, and any combination thereof.

82. The oral dosage form of claim 81 wherein said at least one solubility enhancer is a macrogol fatty acid ester.

83. The oral dosage form of claim 82 wherein said macrogol fatty acid ester is in an amount greater than from about 0% to about 95% by weight of each microparticle.

84. The oral dosage form of claim 83 wherein said macrogol fatty acid ester is present in an amount from about 1% to about 50% by weight of each microparticle.

85. The oral dosage form of claim 84 wherein said macrogol fatty acid ester is present in an amount from about 5% to about 35% by weight of each microparticle.

86. The oral dosage form of claim 85 wherein said macrogol fatty acid ester is present in an amount of about 5% by weight of each microparticle.

87. The oral dosage form of claim 85 wherein said macrogol fatty acid ester is present in an amount of about 35% by weight of each microparticle.

88. The oral dosage form of claim 49 wherein said dosage form is incorporated into a tablet.

89. An oral fast-dispersing dosage form comprising:
(a) a plurality of microparticles comprising a rapid absorption composition of an effective amount of sumatriptan, glyceryl palmitostearate, and macrogol fatty acid ester, wherein said sumatriptan is present in an amount of about 30% by weight of a microparticle, said glyceryl palmitostearate is present in an amount of about 65% by weight of a microparticle and said macrogol fatty acid ester is present in an amount of about 5% by weight of a microparticle, said microparticles coated with at least one-taste masking coating,
(b) a non-cushioning matrix comprising mannitol, microcrystalline cellulose, and crospovidone, wherein said mannitol is present in an amount of about 45%, said microcrystalline cellulose is present in an amount of about 15%, and said crospovidone is present in an amount of about 2% by weight of said dosage form, and
(c) pharmaceutically acceptable excipients,
wherein said taste-masked coated microparticles are dispersed within said matrix.

90. The oral fast-dispersing dosage form of claim 89 wherein said dosage form when administered to a patient in need of such administration exhibits a blood absorption profile such that after about 0.5 hours at least about 15% of the sumatriptan is absorbed, after about 0.75 hours at least about 35% of the sumatriptan is absorbed, after about 1 hour at least about 50% of the sumatriptan is absorbed, after about 1.5 hours at least about 70% of the sumatriptan is absorbed, after about 2 hours at least about 80% of the sumatriptan is absorbed, after about 4 hours at least about 90% of the sumatriptan is absorbed, and after about 6 hours at least about 95% of the sumatriptan is absorbed into the blood stream of the patient.

91. The oral fast-dispersing dosage form of claim 89 wherein said dosage form when administered to a patient in need of such administration exhibits a mean sumatriptan blood absorption profile as shown in FIG. 5A.

92. The oral fast-dispersing dosage form of claim 89 Wherein said dosage form when administered to a patient in need of such administration provides a Tₘₐₓ from about 1 hour to about 3 hours and a Cₘₐₓ of about 15 ng/ml to about 46 ng/ml sumatriptan in the blood after administration of a 50 mg sumatriptan dosage form to the patient.

93. The oral fast-dispersing dosage form of claim 89 wherein said dosage form when administered to a patient in need of such administration provides a mean Tₘₐₓ of about 1.7 hours and a mean Cₘₐₓ of about 28 ng/ml sumatriptan in the blood after administration of a 50 mg sumatriptan dosage form to the patient.

94. The oral fast-dispersing dosage form of claim 89 wherein said dosage form when administered to a patient in need of such administration exhibits a plasma profile as shown in FIG. 4A for a 50 mg sumatriptan dosage form.

95. An oral fast-dispersing dosage form comprising:
(a) a plurality of microparticles comprising a rapid absorption composition of an effective amount of sumatriptan, glyceryl palmitostearate, and macrogol fatty acid ester, wherein said sumatriptan is present in an amount of about 30% by weight of a microparticle, said glyceryl palmitostearate is present in an amount of about 35% by weight of a microparticle and said macrogol fatty acid ester is present in an amount of about 35% by weight of a microparticle, said microparticles coated with at least one-taste masking coating;
(b) a non-cushioning matrix comprising mannitol, microcrystalline cellulose, and crospovidone, wherein said mannitol is present in an amount of about 45%, said microcrystalline cellulose is present in an amount of about 15%, and said crospovidone is present in an amount of about 2% by weight of said dosage form, and
(c) pharmaceutically acceptable excipients,
wherein said taste-masked coated microparticles are dispersed within said matrix.

96. The oral fast-dispersing dosage form of claim 95 wherein said dosage form when administered to a patient in need of such administration exhibits a blood absorption profile such that after about 0.5 hours at least about 20% of the sumatriptan is absorbed, after about 0.75 hours at least about 40% of the sumatriptan is absorbed, after about 1 hour at least about 55% of the sumatriptan is absorbed, after about 1.5 hours at least about 76% of the sumatriptan is absorbed, after about 2 hours at least about 80% of the sumatriptan is absorbed, after about 4 hours at least about 90% of the sumatriptan is absorbed, and after about 6 hours at least about 95% of the sumatriptan is absorbed into the blood stream of the patient.

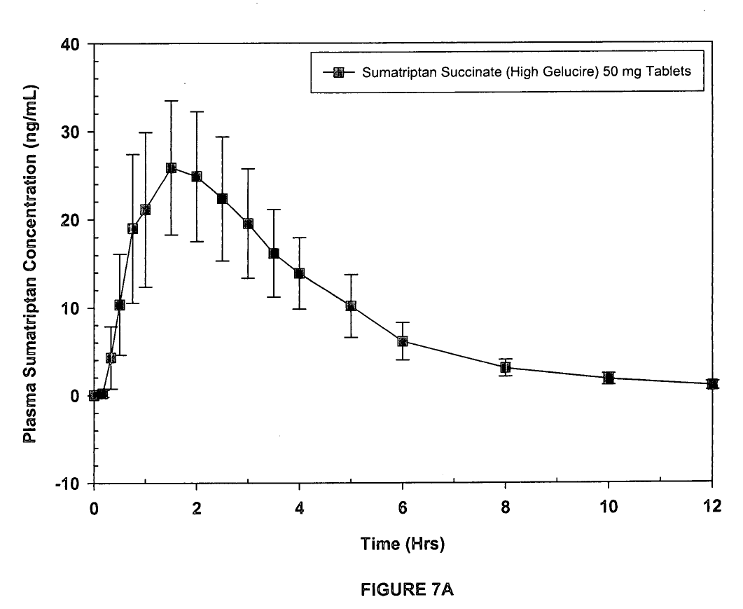

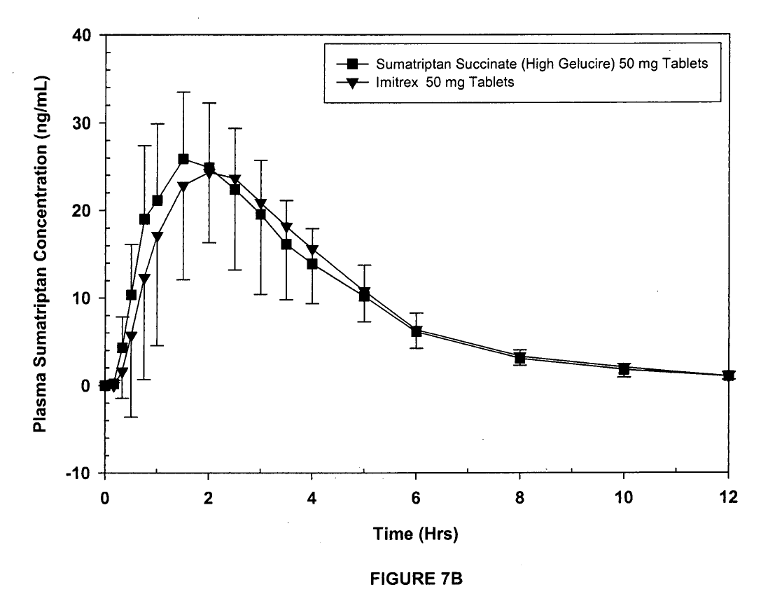

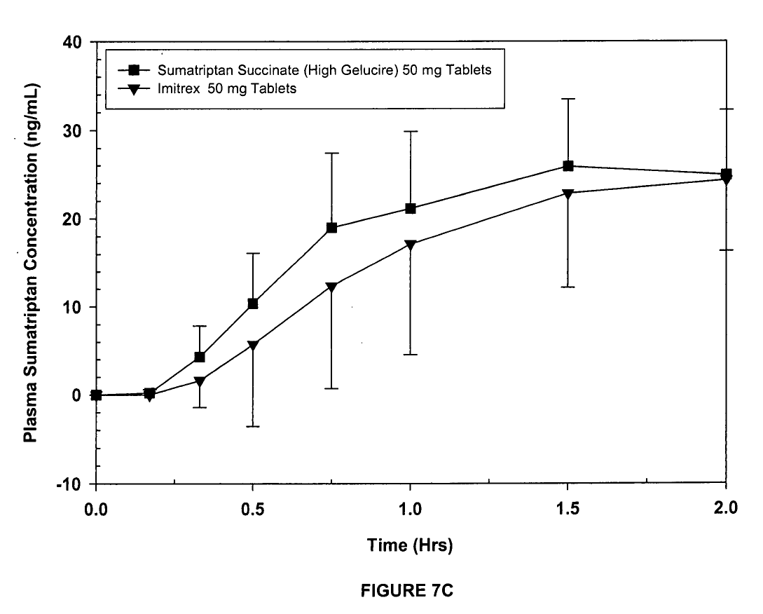

97. The oral fast-dispersing dosage form of claim 95 wherein said dosage form when administered to a patient in need of such administration exhibits a mean sumatriptan blood absorption profile as shown in FIG. 7A.

98. The oral fast-dispersing dosage form of claim 95 wherein said dosage form when administered to a patient in need of such administration provides a Tₘₐₓ from about 0.75 hours to about 2 hours and a Cₘₐₓ of about 14 ng/ml to about 46 ng/ml sumatriptan in the blood after administration of a 50 mg sumatriptan dosage form to the patient.

99. The oral fast-dispersing dosage form of claim 95 wherein said dosage form when administered to a patient in need of such administration provides a mean Tₘₐₓ of about 1.6 hours and a mean Cₘₐₓ of about 27 ng/ml sumatriptan in the blood after administration of a 50 mg sumatriptan dosage form to the patient.

100. The oral fast-dispersing dosage form of claim 95 wherein said dosage form when administered to a patient in need of such administration exhibits a plasma profile as shown in FIG. 6A for a 50 mg sumatriptan dosage form.

101. The oral fast-dispersing dosage form of claim 89 wherein said dosage form when administered to a patient in need of such administration exhibits an AUC₍₀₋ₜ₎ from about 69 ng.hr/ml to about 163 ng.hr/ml for a 50 mg sumatriptan dosage form.

102. The oral fast-dispersing dosage form of claim 89 wherein said dosage form when administered to a patient in need of such administration exhibits a mean AUC₍₀₋ₜ₎ of about 109 ng.hr/ml for a 50 mg sumatriptan dosage form.

103. The oral fast-dispersing dosage form of claim 89 wherein said dosage form when administered to a patient in need of such administration exhibits an AUC_{(0-inf)} from about 70 ng.hr/ml to about 166 ng.hr/ml for a 50 mg sumatriptan dosage form.

104. The oral fast-dispersing dosage form of claim 89 wherein said dosage form when administered to a patient in need of such administration exhibits a mean AUC_{(0-inf)} of about 112 ng.hr/ml for a 50 mg sumatriptan dosage form.

105. The oral fast-dispersing dosage form of claim 95 wherein said dosage form when administered to a patient in need of such administration exhibits an AUC₍₀₋ₜ₎ from about 60 ng.hr/ml to about 165 ng.hr/ml for a 50 mg sumatriptan dosage form.

106. The oral fast-dispersing dosage form of claim 95 wherein said dosage form when administered to a patient in need of such administration exhibits a mean AUC₍₀₋ₜ₎ of about 110 ng.hr/ml for a 50 mg sumatriptan dosage form.

107. The oral fast-dispersing dosage form of claim 95 wherein said dosage form when administered to a patient in need of such administration exhibits an AUC_{(0-inf)} from about 62 ng.hr/ml to about 170 ng.hr/ml for a 50 mg sumatriptan dosage form.

108. The oral fast-dispersing dosage form of claim 95 wherein said dosage form when administered to a patient in need of such administration exhibits a mean AUC_{(0-inf)} of about 114 ng.hr/ml for a 50 mg sumatriptan dosage form.

109. An oral fast-dispersing dosage form comprising:
(a) a plurality of microparticles comprising a rapid absorption composition of an effective amount of sumatriptan, glyceryl palmitostearate, and macrogol fatty acid ester, wherein said sumatriptan is present in an amount of about 30% by weight of a microparticle, said glyceryl palmitostearate is present in an amount of about 65% by weight of a microparticle and said macrogol fatty acid ester is present in an amount of about 5% by weight of a microparticle, said microparticles coated with at least one-taste masking coating,
(b) a non-cushioning matrix comprising mannitol, microcrystalline cellulose, and crospovidone, wherein said mannitol is present in an amount of about 45%, said microcrystalline cellulose is present in an amount of about 15%, and said crospovidone is present in an amount of about 2% by weight of said dosage form, and
(c) pharmaceutically acceptable excipients,
wherein said taste-masked coated microparticles are dispersed within said matrix and wherein said dosage form when administered to a patient in need of such administration exhibits a blood absorption profile such that after about 0.5 hours at least about 15% of the sumatriptan is absorbed, after about 0.75 hours at least about 35% of the sumatriptan is absorbed, after about 1 hour at least about 50% of the sumatriptan is absorbed, after about 1.5 hours at least about 70% of the sumatriptan is absorbed, after about 2 hours at least about 80% of the sumatriptan is absorbed, after about 4 hours at least about 90% of the sumatriptan is absorbed, and after about 6 hours at least about 95% of the sumatriptan is absorbed into the blood stream of the patient.

110. An oral fast-dispersing dosage form comprising:
(a) a plurality of microparticles comprising a rapid absorption composition of an effective amount of sumatriptan, glyceryl palmitostearate, and macrogol fatty acid ester, wherein said sumatriptan is present in an amount of about 30% by weight of a microparticle, said glyceryl palmitostearate is present in an amount of about 65% by weight of a microparticle and said macrogol fatty acid ester is present in an amount of about 5% by weight of a microparticle, said microparticles coated with at least one-taste masking coating,
(b) a non-cushioning matrix comprising mannitol, microcrystalline cellulose, and crospovidone, wherein said mannitol is present in an amount of about 45%, said microcrystalline cellulose is present in an amount of about 15%, and said crospovidone is present in an amount of about 2% by weight of said dosage form, and
(c) pharmaceutically acceptable excipients, wherein said taste-masked coated microparticles are dispersed within said matrix and wherein said dosage form when administered to a patient in need of such administration provides a Tₘₐₓ from about 1 hour to about 3 hours and a Cₘₐₓ of about 15 ng/ml to about 46 ng/ml sumatriptan in the blood after administration of a 50 mg sumatriptan dosage form to the patient.

111. An oral fast-dispersing dosage form comprising:
(a) a plurality of microparticles comprising a rapid absorption composition of an effective amount of sumatriptan, glyceryl palmitostearate, and macrogol fatty acid ester, wherein said sumatriptan is present in an amount of about 30% by weight of a microparticle, said glyceryl palmitostearate is present in an amount of about 65% by weight of a microparticle and said macrogol fatty acid ester is present in an amount of about 5% by weight of a microparticle, said microparticles coated with at least one-taste masking coating,
(b) a non-cushioning matrix comprising mannitol, microcrystalline cellulose, and crospovidone, wherein said mannitol is present in an amount of about 45%, said microcrystalline cellulose is present in an amount of about 15%, and said crospovidone is present in an amount of about 2% by weight of said dosage form, and
(c) pharmaceutically acceptable excipients, wherein said taste-masked coated microparticles are dispersed within said matrix and wherein said dosage form when administered to a patient in need of such administration provides a mean Tₘₐₓ of about 1.7 hours and a mean Cₘₐₓ of about 28 ng/ml sumatriptan in the blood after administration of a 50 mg sumatriptan dosage form to the patient.

112. An oral fast-dispersing dosage form comprising:
(a) a plurality of microparticles comprising a rapid absorption composition of an effective amount of sumatriptan, glyceryl palmitostearate, and macrogol fatty acid ester, wherein said sumatriptan is present in an amount of about 30% by weight of a microparticle, said glyceryl palmitostearate is present in an amount of about 65% by weight of a microparticle and said macrogol fatty acid ester is present in an amount of about 5% by weight of a microparticle, said microparticles coated with at least one-taste masking coating,
(b) a non-cushioning matrix comprising mannitol, microcrystalline cellulose, and crospovidone, wherein said mannitol is present in an amount of about 45%, said microcrystalline cellulose is present in an amount of about 15%, and said crospovidone is present in an amount of about 2% by weight of said dosage form, and
(c) pharmaceutically acceptable excipients, wherein said taste-masked coated microparticles are dispersed within said matrix and wherein said dosage form when administered to a patient in need of such administration exhibits a mean sumatriptan blood absorption profile as shown in FIG. 5A.

113. An oral fast-dispersing dosage form comprising:
(a) a plurality of microparticles comprising a rapid absorption composition of an effective amount of sumatriptan, glyceryl palmitostearate, and macrogol fatty acid ester, wherein said sumatriptan is present in an amount of about 30% by weight of a microparticle, said glyceryl palmitostearate is present in an amount of about 65% by weight of a microparticle and said macrogol fatty acid ester is present in an amount of about 5% by weight of a microparticle, said microparticles coated with at least one-taste masking coating,
(b) a non-cushioning matrix comprising mannitol, microcrystalline cellulose, and crospovidone, wherein said mannitol is present in an amount of about 45%, said microcrystalline cellulose is present in an amount of about 15%, and said crospovidone is present in an amount of about 2% by weight of said dosage form, and
(c) pharmaceutically acceptable excipients, wherein said taste-masked coated microparticles are dispersed within said matrix and wherein said dosage form when administered to a patient in need of such administration exhibits a plasma profile as shown in FIG. 4A for a 50 mg sumatriptan dosage form.

114. An oral fast-dispersing dosage form comprising:
(a) a plurality of microparticles comprising a rapid absorption composition of an effective amount of sumatriptan, glyceryl palmitostearate, and macrogol fatty acid ester, wherein said sumatriptan is present in an amount of about 30% by weight of a microparticle, said glyceryl palmitostearate is present in an amount of about 35% by weight of a microparticle and said macrogol fatty acid ester is present in an amount of about 35% by weight of a microparticle, said microparticles coated with at least one-taste masking coating;
(b) a non-cushioning matrix comprising mannitol, microcrystalline cellulose, and crospovidone, wherein said mannitol is present in an amount of about 45%, said microcrystalline cellulose is present in an amount of about 15%, and said crospovidone is present in an amount of about 2% by weight of said dosage form, and
(c) pharmaceutically acceptable excipients, wherein said taste-masked coated microparticles are dispersed within said matrix and wherein said dosage form when administered to a patient in need of such administration exhibits a blood absorption profile such that after about 0.5 hours at least about 20% of the sumatriptan is absorbed, after about 0.75 hours at least about 40% of the sumatriptan is absorbed, after about 1 hour at least about 55% of the sumatriptan is absorbed, after about 1.5 hours at least about 76% of the sumatriptan is absorbed, after about 2 hours at least about 80% of the sumatriptan is absorbed, after about 4 hours at least about 90% of the sumatriptan is absorbed, and after about 6 hours at least about 95% of the sumatriptan is absorbed into the blood stream of the patient.

115. An oral fast-dispersing dosage form comprising:
(a) a plurality of microparticles comprising a rapid absorption composition of an effective amount of sumatriptan, glyceryl palmitostearate, and macrogol fatty acid ester, wherein said sumatriptan is present in an amount of about 30% by weight of a microparticle, said glyceryl palmitostearate is present in an amount of about 35% by weight of a microparticle and said macrogol fatty acid ester is present in an amount of about 35% by weight of a microparticle, said microparticles coated with at least one-taste masking coating;
(b) a non-cushioning matrix comprising mannitol, microcrystalline cellulose, and crospovidone, wherein said mannitol is present in an amount of about 45%, said microcrystalline cellulose is present in an amount of about 15%, and said crospovidone is present in an amount of about 2% by weight of said dosage form, and
(c) pharmaceutically acceptable excipients, wherein said taste-masked coated microparticles are dispersed within said matrix and wherein said dosage form when administered to a patient in need of such administration provides a Tₘₐₓ from about 0.75 hours to about 2 hours and a Cₘₐₓ of about 14 ng/ml to about 46 ng/ml sumatriptan in the blood after administration of a 50 mg sumatriptan dosage form to the patient.

116. An oral fast-dispersing dosage form comprising:
(a) a plurality of microparticles comprising a rapid absorption composition of an effective amount of sumatriptan, glyceryl palmitostearate, and macrogol fatty acid ester, wherein said sumatriptan is present in an amount of about 30% by weight of a microparticle, said glyceryl palmitostearate is present in an amount of about 35% by weight of a microparticle and said macrogol fatty acid ester is present in an amount of about 35% by weight of a microparticle, said microparticles coated with at least one-taste masking coating;
(b) a non-cushioning matrix comprising mannitol, microcrystalline cellulose, and crospovidone, wherein said mannitol is present in an amount of about 45%, said microcrystalline cellulose is present in an amount of about 15%, and said crospovidone is present in an amount of about 2% by weight of said dosage form, and
(c) pharmaceutically acceptable excipients, wherein said taste-masked coated microparticles are dispersed within said matrix and wherein said dosage form when administered to a patient in need of such administration provides a mean Tₘₐₓ of about 1.6 hours and a mean Cₘₐₓ of about 27 ng/ml sumatriptan in the blood after administration of a 50 mg sumatriptan dosage form to the patient.

117. An oral fast-dispersing dosage form comprising:
(a) a plurality of microparticles comprising a rapid absorption composition of an effective amount of sumatriptan, glyceryl palmitostearate, and macrogol fatty acid ester, wherein said sumatriptan is present in an amount of about 30% by weight of a microparticle, said glyceryl palmitostearate is present in an amount of about 35% by weight of a microparticle and said macrogol fatty acid ester is present in an amount of about 35% by weight of a microparticle, said microparticles coated with at least one-taste masking coating;
(b) a non-cushioning matrix comprising mannitol, microcrystalline cellulose, and crospovidone, wherein said mannitol is present in an amount of about 45%, said microcrystalline cellulose is present in an amount of about 15%, and said crospovidone is present in an amount of about 2% by weight of said dosage form, and
(c) pharmaceutically acceptable excipients, wherein said taste-masked coated microparticles are dispersed within said matrix and wherein said dosage form when administered to a patient in need of such administration exhibits an AUC₍₀₋ₜ₎ from about 69 ng.hr/ml to about 163 ng.hr/ml for a 50 mg sumatriptan dosage form.

118. An oral fast-dispersing dosage form comprising:
(a) a plurality of microparticles comprising a rapid absorption composition of an effective amount of sumatriptan, glyceryl palmitostearate, and macrogol fatty acid ester, wherein said sumatriptan is present in an amount of about 30% by weight of a microparticle, said glyceryl palmitostearate is present in an amount of about 35% by weight of a microparticle and said macrogol fatty acid ester is present in an amount of about 35% by weight of a microparticle, said microparticles coated with at least one-taste masking coating;
(b) a non-cushioning matrix comprising mannitol, microcrystalline cellulose, and crospovidone, wherein said mannitol is present in an amount of about 45%, said microcrystalline cellulose is present in an amount of about 15%, and said crospovidone is present in an amount of about 2% by weight of said dosage form, and
(c) pharmaceutically acceptable excipients, wherein said taste-masked coated microparticles are dispersed within said matrix and wherein said dosage form when administered to a patient in need of such administration exhibits a mean AUC₍₀₋ₜ₎ of about 109 ng.hr/ml for a 50 mg sumatriptan dosage form.

119. An oral fast-dispersing dosage form comprising:
(a) a plurality of microparticles comprising a rapid absorption composition of an effective amount of sumatriptan, glyceryl palmitostearate, and macrogol fatty acid ester, wherein said sumatriptan is present in an amount of about 30% by weight of a microparticle, said glyceryl palmitostearate is present in an amount of about 35% by weight of a microparticle and said macrogol fatty acid ester is present in an amount of about 35% by weight of a microparticle, said microparticles coated with at least one-taste masking coating;
(b) a non-cushioning matrix comprising mannitol, microcrystalline cellulose, and crospovidone, wherein said mannitol is present in an amount of about 45%, said microcrystalline cellulose is present in an amount of about 15%, and said crospovidone is present in an amount of about 2% by weight of said dosage form, and
(c) pharmaceutically acceptable excipients, wherein said taste-masked coated microparticles are dispersed within said matrix and wherein said dosage form when administered to a patient in need of such administration exhibits a plasma profile as shown in FIG. 6A after administration of a 50 mg sumatriptan dosage form.

120. An oral fast-dispersing dosage form comprising:
(a) a plurality of microparticles comprising a rapid absorption composition of an effective amount of sumatriptan, glyceryl palmitostearate, and macrogol fatty acid ester, wherein said sumatriptan is present in an amount of about 30% by weight of a microparticle, said glyceryl palmitostearate is present in an amount of about 35% by weight of a microparticle and said macrogol fatty acid ester is present in an amount of about 35% by weight of a microparticle, said microparticles coated with at least one-taste masking coating;
(b) a non-cushioning matrix comprising mannitol, microcrystalline cellulose, and crospovidone, wherein said mannitol is present in an amount of about 45%, said microcrystalline cellulose is present in an amount of about 15%, and said crospovidone is present in an amount of about 2% by weight of said dosage form, and
(c) pharmaceutically acceptable excipients, wherein said taste-masked coated microparticles are dispersed within said matrix and wherein said dosage form when administered to a patient in need of such administration exhibits a mean sumatriptan blood absorption profile as shown in FIG. 7A.

121. The oral fast-dispersing dosage form of claim 89 wherein said dosage form when administered to a patient provides a plasma concentration-time curve such that the ratio of the T₅₀ of said composition to the T₅₀ of Imitrex is less than about 1.

122. The oral fast-dispersing dosage form of claim 121 wherein the ratio of the AUC₍₀₋ₜ₎ of the composition to the AUC₍₀₋ₜ₎ of Imitrex and the ratio of the Cₘₐₓ of the composition to the Cₘₐₓ of Imitrex is about 1.

123. The oral fast-dispersing dosage form of claim 95 wherein said dosage form when administered to a patient provides a plasma concentration-time curve such that the ratio of the T50 of said composition to the T50 of Imitrex is less than about 1.

124. The oral fast-dispersing dosage form of claim 123 wherein the ratio of the AUC₍₀₋ₜ₎ of the composition to the AUC₍₀₋ₜ₎ of Imitrex and the ratio of the Cₘₐₓ of the composition to the Cₘₐₓ of Imitrex is about 1.

125. The rapid absorption pharmaceutical composition of claim 9 wherein said sumatriptan is present in an amount of about 30% by weight of said microparticle.

126. The rapid absorption pharmaceutical composition of claim 9 wherein said sumatriptan is present in an amount of about 40% by weight of each microparticle.

127. The rapid absorption pharmaceutical composition of claim 13 wherein said distilled glyceryl palmitostearate is present in an amount of about 65% by weight of each microparticle.

128. The rapid absorption pharmaceutical composition of claim 14 wherein said distilled glyceryl palmitostearate is present in an amount of about 35% by weight of each microparticle.

129. The rapid absorption pharmaceutical composition of claim 14 wherein said distilled glyceryl palmitostearate is present in an amount of about 25% by weight of each microparticle.

130. The oral dosage form of claim 75 wherein said sumatriptan is present in an amount of about 30% by weight of said microparticle.

131. The oral dosage form of claim 75 wherein said sumatriptan is present in an amount of about 40% by weight said microparticle.

132. The oral dosage form of claim 79 wherein said glyceryl palmitostearate is present in an amount of about 65% by weight of each microparticle.

133. The oral dosage form of claim 80 wherein said glyceryl palmitostearate is present in an amount of about 35% by weight of each microparticle.

134. The oral dosage form of claim 80 wherein said glyceryl palmitostearate is present in an amount of about 25% by weight of each microparticle.

135. An oral fast-dispersing dosage form comprising:
(a) a plurality of microparticles comprising a rapid absorption composition of an effective amount of sumatriptan, glyceryl palmitostearate, and macrogol fatty acid ester, wherein said sumatriptan is present in an amount of about 40% by weight of a microparticle, said glyceryl palmitostearate is present in an amount of about 25% by weight of a microparticle and said macrogol fatty acid ester is present in an amount of about 35% by weight of a microparticle, said microparticles coated with at least one-taste masking coating,
(b) a non-cushioning matrix comprising mannitol, microcrystalline cellulose, and crospovidone, wherein said mannitol is present in an amount of about 45%, said microcrystalline cellulose is present in an amount of about 15%, and said crospovidone is present in an amount of about 2% by weight of said dosage form, and
(c) pharmaceutically acceptable excipients,
wherein said taste-masked coated microparticles are dispersed within said matrix.
